(19) United States
(12) Patent Application Publication
(10) Pub. No.: US 2026/0345846 A1
(43) Pub. Date: Nov. 5, 2026

(54) COMPOSITIONS

(71) Applicant: Reckitt Benckiser LLC, Nutley, NJ (US)

(72) Inventors: Pamela ASPLUNDH, Montvale, NJ (US); Mohammad Khalid IJAZ, Montvale, NJ (US); Anthony KEVEK, Montvale, NJ (US); Mark MILLER, Montvale, NJ (US); Carrie Ann ROMANOV, Montvale, NJ (US)

(21) Appl. No.: 19/710,997

(22) Filed: Jun. 17, 2026

Related U.S. Application Data
(63) Continuation of application No. PCT/EP2024/087675, filed on Dec. 19, 2024.
(60) Provisional application No. 63/612,571, filed on Dec. 20, 2023.

(30) Foreign Application Priority Data
Mar. 11, 2024 (GB) 2403478.7

(51) Int. Cl. A61L 2/22; A61L 2/20; A61L 101/34; A61L 101/38; A61L 101/50

(52) U.S. Cl. CPC A61L 2/22; A61L 2/20; A61L 2101/34; A61L 2101/38; A61L 2101/50

(57) ABSTRACT

Compositions are disclosed comprising 7-43% wt dipropylene glycol, 28-50% wt alcohol, 30-45% wt water, and 0.2-2% sheer fragrance, wherein the sheer fragrance comprises approximately 10% wt to approximately 80% wt top notes based on the total weight of the sheer fragrance. Products are also disclosed comprising the disclosed compositions in an aerosol can comprising approximately 60 psig or more of a pressurizing gas. The disclosed compositions reduce or suppress the perception of malodour in the air. The disclosed compositions also sanitize or disinfect a volume of air less than or equal to 25 m³ in 0.1 to 5 minutes by providing equal to or greater than a 3 log₁₀ reduction of aerosolized bacteria, including Staphylococcus aureus and Klebsiella pneumoniae, and aerosolized enveloped viruses, including Phi6. A 3 log₁₀ reduction of aerosolized non-enveloped viruses, like MS2, occurs in 10-15 minutes.

### PRIORITY

This application is a continuation-in-part filed pursuant to 35 USC § 111(a) of PCT application number PCT/EP2024/087675 filed 19 Dec. 2024, which claims priority to GB application number 2403478.7, filed 11 Mar. 2024, and U.S. provisional application No. 63/612,571, filed 20 Dec. 2023, the contents of all three priority applications being incorporated herein by reference thereto in their entireties.

### TECHNICAL FIELD

Compositions are disclosed comprising 7-43% wt dipropylene glycol, 28-50% wt alcohol, 30-45% wt water, and 0.2-2% sheer fragrance, all based on the weight of the composition, wherein the sheer fragrance comprises approximately 10% wt to approximately 80% wt top notes based on the total weight of the sheer fragrance. Products are also disclosed comprising the disclosed compositions in an aerosol can comprising approximately 60 psig or more of a pressurizing gas. The disclosed compositions reduce or suppress the perception of malodour in the air. The disclosed compositions also sanitize or disinfect a volume of air less than or equal to 25 m³ in 0.1 to 5 minutes by providing equal to or greater than a 3 log₁₀ reduction of aerosolized bacteria, including Staphylococcus aureus and Klebsiella pneumoniae, and aerosolized enveloped viruses, including Phi6. A 3 log₁₀ reduction of aerosolized non-enveloped viruses, like bacteriophage MS2 (Emnesvirus zinderi), commonly called MS2, occurs in 10-15 minutes.

### BACKGROUND

Exposure to respiratory droplets from an infected subject in the air or on solid surfaces can be an important vehicle for spread of a variety of human pathogens, as evidenced by the recent COVID-19 pandemic associated with severe acute respiratory coronavirus-2 (SARS-CoV-2) (see, e.g., Ijaz et al., PeerJ, DOI 10.7717/peerj.16420 (2023)).

U.S. Pat. No. 2,719,129 to Richardson et al. discloses liquid room and air deodorant compositions comprising 0.5-5% of a quaternary morpholinium alkyl sulfate having an alkyl radical containing 8 to 24 carbon atoms as a deodorant, about 80-95% of a liquefied normally gaseous low molecular weight halogenated hydrocarbon propellent, and a sufficient amount of a partial ester of a polyhydric alcohol and a higher fatty acid having about 8 to 24 carbon atoms.

CN Pat App Pub No 108324983 discloses an air freshener for removing odour and a preparation method thereof.

US Pat App Pub No 2020/068878 to Clorox Co discloses, in paragraph 0056, a spray composition for killing bedbugs and sterilizing surfaces comprising 5.31% dipropylene glycol, 66% ethanol, 23% water, and 5.25% propane, d-limonene, and sodium nitrite.

GB Pat 2,608,465 to Reckitt Benckiser LLC discloses air decontamination formulations comprising 28-38% by weight dipropylene glycol, 0.12-0.37% by weight odour neutralizing cationic surfactant, 28-50% by weight alcohol, and 14-33% by weight water.

U.S. Pat. No. 11,801,212 to Thnktnk LLC discloses a composition for reducing unpleasant odour comprising betaine, musk, and N-soya-n-ethyl morpholinium ethosulfate.

A need remains to develop microbicidal compositions demonstrating efficacy against airborne pathogens that also exhibit malodour control.

### BRIEF SUMMARY

Compositions are disclosed comprising approximately 7% wt to approximately 43% wt dipropylene glycol; approximately 28 wt to approximately 50% wt alcohol; approximately 30% wt to approximately 45% wt water; and approximately 0.2% wt to approximately 2% wt sheer fragrance, all based on the total weight of the composition, wherein the sheer fragrance comprises approximately 10% wt to approximately 80% wt top notes based on the total weight of the sheer fragrance. The disclosed compositions reduce or suppress the perception of malodour in the air. The disclosed compositions also sanitize or disinfect a volume of air less than or equal to 25 m³ in 0.1 to 5 minutes by providing equal to or greater than a 3 log₁₀ reduction of aerosolized bacteria, including Staphylococcus aureus and Klebsiella pneumoniae, and aerosolized enveloped viruses, including Phi6. A 3 log₁₀ reduction of aerosolized non-enveloped viruses, like MS2, occurs in 10-15 minutes.

Aerosol products containing the disclosed compositions are also disclosed.

Air treatment methods utilizing the disclosed compositions and/or aerosol products are also disclosed.

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 is a diagram of the 2-piece MB nozzle aerosol valve assembly that may be used in the present invention;

FIG. 2 is a perspective view of an aerobiological testing chamber used in the examples that follow;

FIG. 3 is a flow chart of the steps performed in the following examples;

FIG. 4 is a graph of the log₁₀ concentration of the tested microorganism versus time;

FIG. 5 is a graph of the log₁₀ concentration of the tested microorganism versus time;

FIG. 6 is a graph of the log₁₀ concentration of the tested microorganism versus time;

FIG. 7 is a graph of the log₁₀ concentration of the tested microorganism versus time;

FIG. 8 is a graph of the log₁₀ concentration of the tested microorganism versus time;

FIG. 9 is a diagram of the OVS-7 sorbent tube used to sample the concentration of DPG in the air;

FIG. 10 is an exemplary calibration chromatogram showing the elution time for the three (3) different concentrations of DPG standards, including the peaks for the DPG isomers;

FIG. 11 is a graph of the concentration of DPG in air in ppm versus time in minutes.

### DEFINITIONS

As used herein, the term “a” or “an” means one or more.

As used herein, the term “comprising” is inclusive or open-ended and does not exclude any additional elements; the term “consisting of” excludes any additional elements; and the term “consisting essentially of” is in-between, only permitting additional elements that do not materially affect characteristics of the product or process.

As used herein, the term “approximately” or “about” means plus or minus 10 percent of the value stated.

As used herein, the w/w percent of an ingredient is based on the weight of the ingredient in grams in the total weight of the composition in grams. When a raw material does not contain close or equal to 100% active material, two percentages may be provided: one for the weight of the raw material and one for the weight of the active. For example, 0.2 g of Forestall-LQ-(HM) sold by Croda contains approximately 35% soyethyl morpholinium ethosulfate in water, alcohol and/or polypropylene glycol, which equates to approximately 0.07% of soyethyl morpholinium ethosulfate in the composition. This would be represented as 0.2% [0.07%] of odour neutralizing cationic surfactant.

As used herein, any and all ranges are inclusive of their endpoints regardless of the leading preposition. For example, a pH ranging from 9 to 11 or a pH between 9 and 11 would include compositions having a pH of 9, compositions having a pH of 11, and compositions having any pH between 9 and 11.

As used herein, the phrase “substantially free” means a concentration of less than 0.2 g/L, preferably less than 0.1 g/L, more preferably less than 0.05 g/L, and most preferably less than 0.001 g/L.

As used herein, the terms “germ” and “microbe” or “microbial” means microorganisms which causes disease and encompasses both bacteria and viruses and “microbicidal” means compositions that inactivate (or kill) germs and microbes.

As used herein, the terms “decontamination” or “decontaminate” mean to reduce the concentration of aerosolized microbes in the air. The terms “sanitize”, “disinfect,” “sanitization,” and “disinfection” mean providing equal to or greater than a 3 log₁₀ reduction in 0.1 to 5 minutes of aerosolized bacteria, including Staphylococcus aureus and Klebsiella pneumoniae, and aerosolized enveloped viruses, including Phi6, in air occupying a volume less than or equal to 25 m³. The terms “sanitize”, “disinfect,” “sanitization,” and “disinfection” also mean providing equal to or greater than a 3 log₁₀ reduction in 0.1 to 15 minutes of non-enveloped viruses, such as MS2.

As used herein, the term “vapor” refers to the gaseous state of DPG. The term vapor does not include any droplets suspended in the air, but may be formed from such droplets.

As used herein, the term “swirl chamber” may also be known as a spin chamber.

As used herein, the term “sheer fragrance” refers to a fragrance comprising more top notes than bottom notes.

As used herein, the term “aroma chemical” means a chemical substance that provides a scent.

As used herein, the term “top note” refers to aroma chemicals having a boiling point less than or equal to 250 °C when measured at 1 atm pressures, preferably less than 225 °C, more preferably less than 200 °C. Top notes may also be referred to as head notes or opening notes. Exemplary top note aroma chemicals may be described as citrus, delicate, fresh, fruity, green, or light.

As used herein, the term “bottom note” refers to aroma chemicals having a boiling point greater than to 250 °C when measured at 1 atm pressures, preferably greater than 275 °C, more preferably 300 °C. Bottom notes may also be referred to as base notes. Exemplary bottom note aroma chemicals may be described balsamic, floral heavy, musk, spicy, sweet, or woody.

As used herein, the term “pro-fragrance” refers to a perfume compound which is able to release one or more aroma chemicals when triggered by an external influence. Release of the aroma chemical prolongs the perfuming effect. The release may be triggered by exposure to light, air, oxygen, heat, moisture, an enzyme, or any combination thereof.

As used herein, the term “hang time” refers to the amount of time during which the vaporized air decontamination and malodour control compositions remains dispersed in the air.

### DETAILED DESCRIPTION

Applicant has developed a formulation that provides a 99.9% reduction of viruses and bacteria in air. See, e.g., GB Pat No 2,608,465. The initial disclosure focused on finding the balance of ingredients needed to obtain germ kill in the air. A need remains to improve other properties of the formulation, such as malodour control, and to provide a longer lasting fragrance.

In GB Pat No 2,608,465, Applicant believed that that air decontamination compositions comprising approximately 28% w/w to approximately 43% w/w dipropylene glycol (DPG), approximately 0.12% w/w to approximately 0.43% w/w odour neutralizing cationic surfactant, approximately 28% w/w to approximately 50% w/w alcohol, and approximately 14% w/w to approximately 33% water were required to provide air disinfection. The original theory was that “more is better” and higher concentrations of DPG were necessary to achieve germ kill.

Applicant has since discovered that the initial concentration of DPG vapor in the air is more important than the concentration of DPG in the composition. More particularly, as shown in the examples that follow, germ kill is obtained when the concentration of DPG vapor in the air ranges from approximately 5 ppm to approximately 30 ppm, preferably from approximately 9 ppm to approximately 12 ppm. This concentration is much higher than the theoretical saturation level of 2-3 ppm expected from DPG vapors and substantially higher than the 50% of the theoretical saturation level (i.e., 1-1.5 ppm) recommended by the EPA in DIS/TSS-11 from 3 Sep. 1980. As a result, the DPG vapor concentration in the disclosed compositions may be as low as approximately 7% wt, provided that the delivery mechanism generates between approximately 5 ppm to approximately 30 ppm DPG in vapor form. The disclosed compositions comprise approximately 7% wt to approximately 43% wt DPG, preferably approximately 10% wt to approximately 25% wt, more preferably approximately 15% wt to approximately 20% wt.

DPG is a colorless, nearly odourless liquid. DPG has a boiling point of 227 °C, a density of 1.02 g/mL at 20 °C, and a viscosity of 75.0 cPs at 25 °C. DPG has a vapor pressure of 2.7 Pa at 20 °C. DPG's vapor pressure increases to about 250 Pa just below 100 °C. DPG is soluble in ethanol and water. DPG is a mixture of three isomeric chemical compounds: 4-oxa-2,6-heptandiol, 2-(2-hydroxy-propoxy)-propan-1-ol, and 2-(2-hydroxy-1-methyl-ethoxy)-propan-ol. One of ordinary skill in the art will recognize that different isomers may have different properties. Applicant has found that DPG produced by non-catalytic hydration reaction with propylene oxide and water followed by vacuum distillation produces a consistent isomer profile.

DPG is a common solvent for fragrances. As a result, Applicant believes that the sheer fragrance used in the disclosed compositions may be suspended longer in the air, resulting in perception of the aroma chemicals for a longer period of time. Hang time refers to the time during which the vaporized air decontamination and malodour control compositions remains dispersed in the air. As shown in the examples that follow, analytical testing has demonstrated that DPG remains detectable in the air for at least 20 minutes. Applicant believes that the sheer fragrance of the disclosed compositions will have a hang time and be detectible in the air for at least 20 minutes, up to approximately 1 hour, preferably up to approximately 2 hours, and more preferably approximately 6 hours. While the fragrance may include DPG, the total concentration of DPG in the disclosed formulations does not include any amounts contributed by the fragrance.

As stated above, DPG is soluble in both water and ethanol. The percentages of water and alcohol in the disclosed air decontamination and malodour control compositions are important to obtain optimal vaporization of the DPG. The disclosed products contain (a) a combination of between approximately 28% wt to approximately 50% wt alcohol and approximately 45% wt to approximately 30% wt water and (b) a suitable dispensing device. In aerosol dispensing applications using a propellant, a combination of between approximately 38% wt to approximately 48% wt alcohol and approximately 30% wt to approximately 40% wt water is particularly preferred, more preferably between approximately 40% wt to approximately 45% wt alcohol and approximately 35% wt to approximately 40% wt water. The composition contains a weight ratio of alcohol:water ranging from approximately 1:0.65 to approximately 1:1.15 for aerosol applications, preferably from approximately 1:0.65 to 1:0.74. The composition contains a weight ratio of DPG:water:alcohol ranges from approximately 1:2:2.3 to approximately 1:1.8:2.5. One of ordinary skill in the art will recognize that the propellant helps to both dry the droplets being dispensed and aerosolize the composition.

The disclosed compositions comprise approximately 28% wt to approximately 50% wt alcohol. Due to its safety profile, ethanol is the preferred alcohol. However, a combination of C1-C6 alcohols may be used in the disclosed compositions without departing from the teachings herein. For example, the quantity of alcohol in the composition may comprise 95% wt ethanol and 5% wt tert-butyl alcohol or 98% wt ethanol and 2% wt isopropanol (e.g., approximately 14% wt of the composition to approximately 47% wt of the composition ethanol and approximately 1% wt of the composition to approximately 3% wt of the composition t-butyl alcohol). One of ordinary skill in the art will recognize that the alcohol may contain a bitterant to try to prevent accidental ingestion of the disclosed composition, such as denatonium benzoate or denatonium saccharide. Alternatively, the alcohol may be substantially free of a bitterant because accidental access to the composition in an aerosol can is unlikely. One of ordinary skill in the art will recognize that both alcohols and propellants are volatile organic compounds (VOCs) and therefore the concentrations of both are also selected to remain within any governmental VOC limits.

The disclosed compositions comprise approximately 30% wt to approximately 45% wt water. Any water type may be used without departing from the teachings herein. However, any metal impurities contained in the tap or distilled water may cause corrosion of the aerosol can. As a result, the water is preferably deionized water.

The disclosed air decontamination and malodour control compositions may further comprise a pH adjuster to maintain a pH ranging from approximately 9 to approximately 11, preferably from approximately 10 to approximately 10.5. One of ordinary skill in the art will recognize that alkaline formulations are required for compatibility with aerosol cans. Exemplary pH adjusters include but are not limited to inorganic bases, alkanolamines, or combinations thereof. Exemplary inorganic bases include hydroxides, such as sodium hydroxide, potassium hydroxide, magnesium hydroxide, ammonium hydroxide, or combination thereof. Exemplary alkanolamines include monoethanolamine, diethanolamine triethanolamine, methylethylhydroxypropylhydroxylamine, or combinations thereof. Buffers may also be used as the pH adjuster, such as glycine and sodium hydroxide, ammonia and ammonium chloride, sodium dihydrogen phosphate and sodium hydroxide, or combinations thereof. The alkanolamines, preferably monoethanolamine, are particularly preferred because they may also help reduce or suppress the rotten egg aroma of hydrogen sulfide, which further enhances the malodour control properties of the disclosed compositions.

The original formulation disclosed in GB Pat 2,608,465 to Reckitt Benckiser LLC contained 0.07% wt active to approximately 0.43% wt active of an odour neutralizing cationic surfactant, preferably from approximately 0.11% wt active to approximately 0.21% wt active. The odour neutralizing cationic surfactant was included in the composition to suppress the alcohol and amine aromas. The “base” composition containing the odour neutralizing cationic surfactant is close to odourless, minimizing any interference from the formulation with the aroma chemicals. Applicant believes that the odour neutralizing cationic surfactant also provides the disclosed compositions with malodour control capabilities. As shown in the examples that follow, the compositions containing the odour neutralizing cationic surfactant successfully reduced the perception of malodour in the air. Applicant further believes that the odour neutralizing cationic surfactant may suppress some of the fragrance notes, particularly the top notes. As a result, the disclosed compositions may contain 0% odour neutralizing cationic surfactants, provided the fragrance used in the formulation successfully masks the alcohol and amine aromas inherent in the formulation.

Alternatively, a higher concentration of the odour neutralizing cationic surfactant than originally disclosed in GB Pat 2,608,465 to Reckitt Benckiser LLC may be needed to better reduce or suppress malodour in the air. Applicant believes that odour molecules in air are harder to remove than on solid surfaces and, as a result, compositions having higher concentrations of odour neutralizing cationic surfactant are needed. More specifically, Applicant believes that a higher concentration of the odour neutralizing cationic surfactant is required for use in air, as opposed to on hard or soft surfaces, such as countertops or furniture. The disclosed compositions may comprise from approximately 0.1% wt to approximately 5% wt of an odour neutralizing cationic surfactant, preferably from approximately 1% wt to approximately 2% wt. Alternatively, the disclosed compositions comprise from 0.5% wt to 1% wt of an odour neutralizing cationic surfactant. Exemplary odour neutralizing cationic surfactants include, but are not limited to, quaternary morpholinium alkyl sulfate compounds, such as soyethyl morpholinium ethosulfate, cetethyl morpholinium ethosulfate, N-myristyl-N-methyl morpholinium methyl sulfate, N-oleyl-N-methyl morpholinium methyl sulfate, or combinations thereof. Soyethyl morpholinium ethosulfate and/or cetyl ethyl morpholinium ethosulfate are particularly preferred odour neutralizing cationic surfactants. Exemplary commercial sources of soyethyl morpholinium ethosulfate include the product sold under the tradename Forestall-LQ-(HM) by Croda. Exemplary commercial sources of cetyl ethyl morpholinium ethosulfate include the product sold under the tradename Barquat cme-A by Lonza.

In another alternative, the disclosed compositions may comprise from approximately 0.1% wt to approximately 5% wt of odour trapping oligosaccharides, including but not limited to alpha-cyclodextrin, beta-cyclodextrin, gamma-cyclodextrin, methyl esters of beta-cyclodextrin, hydroxypropyl beta-cyclodextrin, methylated beta-cyclodextrin, hydroxyethyl alpha-cyclodextrin, hydroxyethyl beta-cyclodextrin, methylated beta-cyclodextrin, or any combination thereof, preferably from approximately 1% wt to approximately 2% wt or from approximately 0.5% wt to approximately 1% wt. Beta-cyclodextrin is commercially available from Carbosynth Ltd as OC06646. Hydroxypropyl beta-cyclodextrin is commercially available from Wacker as Cavasol® W7 HP.

The disclosed compositions comprise approximately 0.2% wt to approximately 2% wt sheer fragrance, preferably approximately 0.2% wt to approximately 1.5% wt, more preferably approximately 0.5% wt to approximately 1% wt. The sheer fragrance comprises more top notes than bottom notes, providing an airy and fresh scent to the composition. Applicant believes that the concentration of DPG in the disclosed compositions will permit perception of the sheer fragrance for longer in the air. More specifically, Applicant believes and is testing the length of time, or hang time, that the sheer fragrance is detectable in the air. Applicant believes that sheer fragrance may be detectible in the air immediately after spraying (i.e., from less than 1 second) to up to approximately 1 hour, preferably up to approximately 2 hours, and more preferably up to approximately 6 hours.

One of ordinary skill in the art will recognize the challenge of fragrancing an alkaline aerosolized composition containing high percentages of alcohol. The propellants amplify the chemical aroma of the ethanol-containing formulation. One of ordinary skill in the art will further recognize that most aroma chemicals are stable at neutral pHs and less stable at the acidic and basic pH extremes. As a result, there are also fewer aroma chemicals that remain stable at the basic pH of the disclosed compositions, producing additional fragrancing complications. Finally, as discussed above, the odour neutralizing cationic surfactant may suppress some of the fragrance notes, particularly the top notes. At higher concentrations of the odour neutralizing cationic surfactant, Applicant believes that using a fragrance containing more top notes than bottom notes will provide a composition that reduces or suppresses both the alcohol and amine aromas of the composition as well as malodour in the air, while remaining airy and fresh scented.

The sheer fragrance comprises between approximately 1% wt to less than approximately 50% wt volatile solvent based on the total weight of the sheer fragrance. While not exhaustive due to the large number of solvent options, exemplary volatile solvents include, but are not limited to, acetone, dipropylene glycol, ethanol, isopropyl alcohol, isopropyl myristate, t-butyl alcohol, or any combinations thereof. To improve stability and promote a uniform dispersion of the sheer fragrance in the air, the solvents in the sheer fragrance preferably comprise the same components contained in the composition, specifically C1-C4 alcohols, dipropylene glycol, or any combination thereof. Applicant believes that the perception of the sheer fragrances will last longer for sheer fragrances comprising C1-C4 alcohols and/or dipropylene glycol solvents. Due to the low concentration of fragrance in the disclosed composition, any DPG and/or ethanol solvent in the fragrance will be negligible and is not included in the total concentrations of these ingredients in the composition.

The sheer fragrance contains between approximately 10% wt to approximately 80% wt top notes based on the total weight of the fragrance, preferably between approximately 15% wt to approximately 65% wt, and more preferably between approximately 25% wt to approximately 50% wt. Applicant has defined top notes as aroma chemicals having a boiling pointless than or equal to 250 °C when measured at 1 atm pressures, preferably less than 225 °C, more preferably less than 200 °C. Applicant has defined bottom notes as aroma chemicals having a boiling point greater than to 250 °C when measured at 1 atm pressures, preferably greater than 275 °C, more preferably 300 °C. One of ordinary skill in the art would be able to identify aroma chemicals meeting this criteria using publicly available literature.

While not exhaustive due to the large number of top notes available, exemplary top notes include, but are not limited to, adoxal, allyl cyclohexane propionate, alpha pinene, alpha terpineol, alpha thujone, benzaldehyde, benzyl alcohol, beta gamma hexenol, beta pinene, borneol, bornyl acetate, camphor, carvacrol, carvone, cis-3-hexenol, cis-3-hexenyl acetate, citral, citronellal nitrate, citronellol, citronellyl acetate, decyl alcohol, dimethyl acetal, dimethyl benzyl carbinol, 2,6-dimethyl-5-hepten-1-al, eucalyptol, geraniol, geranyl acetate, geranyl nitrile, hexyl acetate, hydroquinone dimethyl ether, hydroxycitronellal, iso-amyl acetate, iso-amyl alcohol, limonene, linalool, linalyl acetate, menthol, menthone, methyl heptenone, octanol, phenyl acetaldehyde, phenyl ether alcohol, phenyl ethyl dimethyl carbinol, phenyl propyl alcohol, prenyl acetate, rose oxide cis or trans, thymol, tetrahydrolinalool, triplal, verdox, or any combinations thereof.

While not exhaustive due to the large number of bottom notes available, exemplary bottom notes include, but are not limited to, aldrone, ambroxan, benzophenone, benzyl benzoate, benzyl cinnamate, benzyl phenyl acetate, cepionate, cetalox, citronellyl ethoxalate, cresyl caprylate para, cresyl phenyl acetate para, cyclohexal, diethyl phthalate, dione, dodecalaclactone delta, dodecalactone gamma, ethyl maltol, ethyl vanillin, eugenyl phenyl acetate, evernyl, fixolide, geranyl phenyl acetate, geranyl tiglate, hedione, hexyl cinnamic aldehyde, hexyl salicylate, hyacinth acetals, laitone, linalyl benzoate, linalyl cinnamate, linalyl phenyl acetate, methyl cedryl ketone, rosacetol timberol, undecalactone, vanillin, or any combinations thereof.

One of ordinary skill in the art will recognize that each of these aroma chemicals has a different boiling point at 1 atm pressure, notwithstanding the “top note” or “bottom note” designation. As a result, each aroma chemical will exhibit a different evaporation profile. A fragrance containing more bottom notes than top notes will have less impact on a person entering a room than a fragrance containing more top notes. To provide an improved sensory experience from the disclosed compositions, the sheer fragrance requires more top notes than bottom notes.

The sheer fragrance may further comprise a pro-fragrance. Applicant has defined a pro-fragrance as a perfume compound which is able to release one or more aroma chemicals when triggered by an external influence. Release of the aroma chemical prolongs the perfuming effect. The release may be triggered by exposure to light, air, oxygen, heat, moisture, an enzyme, or any combination thereof. Exemplary pro-fragrance perfume compounds include but are not limited to 3-(dodecylthio)-1-(2,6,6-trimethylcyclohex-3-en-1-yl)butan-1-one sold by Firmenich under the trade name HaloScent™ D, 4-(dodecylthio)-4-(2,6,6-trimethylcyclohex-2-en-1-yl)butan-2-one sold by Firmenich under the trade name HaloScent™ I, 4-(dodecylthio)-4-(2,6,6-trimethylcyclohex-1-en-1-yl)butan-2-one sold by Firmenich under the trade name HaloScent™ I, ethyl N,S-bis(4-oxo-4-(2,6,6-trimethylcyclohex-3-en-1-yl)butan-2-yl)cysteinate sold by Givaudan under the tradename Scentaurus™ Berry, ethyl (Z)-2-acetyl-4-methyltridec-2-enoate sold by Givaudan under the tradename Scentaurus™ Clean, 4-(dodecylthio)-4-methylpentan-2-one sold by Givaudan under the tradename Scentaurus™ Juicy, or any combination thereof.

The sheer fragrance may comprise additional materials standard in the fragrance industry including but not limited to viscosity agents, stabilizing agents, coloring agents, preservatives, fixatives, insect repellants, or any combination thereof.

The sheer fragrance is substantially free of any encapsulated fragrances. Existing encapsulated fragrances are unlikely to be stable in the disclosed composition due to the high concentration of ethanol. Additionally, encapsulated fragrances are likely to rupture going through the actuator during the spraying process.

Applicant believes that the disclosed compositions comprising sheer fragrances with more top notes than bottom notes and either 0% wt or approximately 0.1% wt to approximately 5% wt odour neutralizing cationic surfactant will provide improved malodour control and a longer lasting fragrance. Applicant further believes that use of the disclosed compositions will reduce or suppress the perception of malodour in the air, particularly kitchen malodours, such as fish or bacon, or the chemicals that cause kitchen odors, such as butylamine, dipropyle sulfide, or combinations thereof, bathroom malodours, such as indole, 3-methyl-2-butenal, or combinations thereof, pet malodours, such as dimethyltrisulphide, dimethylsulphie, skatole (also known as 3-methylindole), or combinatios thereof, or mold and mildew malodours, such as species of any of Fusarium, Cladosporium, Penicillium, Aspergillus, or any combination thereof. Applicant further believes that the disclosed compositions will reduce the concentration of microbes in the air in 0.1 to 15 minutes, preferably providing a 3 log₁₀ reduction of bacteria and enveloped viruses in 0.1 to 5 minutes and non-enveloped viruses in 0.1 to 15 minutes.

Methods of determining malodour control and fragrance strength are known. ASTM International, formerly known as the American Society for Testing and Materials (ASTM), develops test methods used in many industries. ASTM E1697-05 (2020), titled Standard Test Method for Unipolar Magnitude Estimation of Sensory Attributes, is used to measure and compare the intensities of attributes, such as fragrance or malodour. ASTM E1593-21 (2021), titled Standard Guide for Assessing the Efficacy of Consumer Products in Reducing the Perception of Malodour, is used to assess the ability of consumer products to reduce malodour intensity from a control state. Gas chromatography (GC) may also be used to measure headspace using time of flight detection. However, GC results may include results that are not detectible to most noses. Additionally, GC results may include results for malodour chemicals that are masked, but not removed, resulting in a false positive.

One of ordinary skill in the art will recognize that measuring malodour intensity reduction differs from measuring fragrance longevity over time. Malodour intensity reduction may also be tested against a variety of malodours, such as cooking, bathroom, pet, mold, mildew, etc. As shown in the examples, Applicant has initiated testing on malodour intensity reduction of cooking malodour. Additional malodours and fragrance longevity testing will be conducted soon and is expected to confirm Applicant's theory that sheer fragrances containing more high notes than bottom notes provide improved malodour control and a longer lasting fragrance as compared to Applicant's current commercial products.

When provided in a metal or alloy container, the disclosed air decontamination and malodour control compositions may optionally further comprise a corrosion inhibitor. The corrosion inhibitor decreases the reaction between the air decontamination and malodour control composition and the metal or alloy container. The same ingredient may provide both the buffering and corrosion inhibitor capabilities to the composition. The disclosed air decontamination and malodour control compositions may optionally comprise between approximately 0.03% wt to approximately 0.3% wt corrosion inhibitor. Exemplary corrosion inhibitors include but are not limited to sodium hydroxide, sodium benzoate, sodium nitrite, sodium silicate, sodium lauryl sarcosinate, borates, or combinations thereof. Exemplary borate corrosion inhibitors may comprise a mixture of monoethanolamine (MEA) borate and monoisopropanol amine (MIPA) borate. An exemplary suitable commercial source of the MEA/MIPA borate corrosion inhibitor is sold under the tradename Crodacor™ BE by Croda.

Alternatively, the metal or alloy container may include an interior coating that prevents reaction between the metal or alloy container and its contents. Exemplary coatings include but are not limited to silicon oxide coatings.

Aerosol compositions frequently include a defoaming agent to prevent foaming. Defoaming agents may also be used to solubilize other ingredients, such as the fragrance. The disclosed compositions do not produce a lot of foam and therefore the defoaming agent is not mandatory, unless required for a specific fragrance. The disclosed air decontamination and malodour control compositions may optionally comprise between 0% wt to approximately 0.28% wt defoaming agent. Exemplary defoaming agents include but are not limited to PEG-12 Dimethicone. An exemplary suitable commercial source of PEG-12 dimethicone is sold under the tradename Xiameter™ OFX-0193 Fluid by Dow.

The disclosed air decontamination and malodour control compositions may optionally further include dye.

The disclosed compositions are a single liquid phase and therefore do not require shaking by consumers before use. Single phase compositions are also typically easier to manufacture, vaporize and exhibit less stability issues than two phase compositions. There is no criticality to the order of addition of the disclosed composition ingredients. Specifically, the ingredients that produce the disclosed air decontamination and malodour control compositions may be mixed in one pot, reducing manufacturing complexity. The resulting compositions have a water-like viscosity (i.e., approximately 0.89 mPa s at 25 °C).

The compositions disclosed above contain all of the ingredients necessary to obtain germ kill in the air (i.e., DPG, alcohol and water and optionally odour neutralizing cationic surfactant). As a result, the disclosed compositions do not require any added antimicrobial actives. The disclosed compositions are therefore substantially free of cationic biocides, halides, halide-containing compounds, silver, silver-containing compounds, phenols, hydrogen peroxide, sodium hypochlorite, chlorhexidine, quaternary ammonium compounds such as benzalkonium chloride or dialkyl dimethyl ammonium chloride, or any combinations thereof.

The disclosed compositions do not require any emulsifiers or amphoteric or nonionic surfactants because the ingredients combine to form a single liquid phase. Emulsifiers may also produce foam, which is undesirable. For example, the disclosed air decontamination and malodour control compositions are substantially free of partial esters of a polyhydric acid, alcohol or ether, such as glycerine monostearate, sorbitol monostearate, propylene glycol monostearate, or diethylene glycol monostearate. The disclosed air decontamination and malodour control compositions are substantially free of glyceryl dilaurate, ethylene glycol monopalmitate, propylene glycol monolaurate, ethylene glycol monostearate, or propylene glycol monopalmitate. One of ordinary skill in the art will recognize that fragrances may contain trace amounts of these ingredients. However, any trace amounts included in any fragrances are utilized for the fragrance itself and would not provide sufficient quantities to affect the properties of the disclosed air decontamination and malodour control compositions. As a result, the disclosed compositions may contain between 0% wt and approximately 0.1% wt of a combination of any emulsifiers and amphoteric and nonionic surfactants, more preferably between 0% wt and approximately 0.075% wt, and most preferably between 0% wt and approximately 0.05% wt.

A particularly preferred air decontamination and malodour control composition consists of approximately 7% wt to approximately 43% wt of the composition of dipropylene glycol; approximately 28% wt to approximately 50% wt of the composition of an alcohol, preferably ethanol; approximately 30% wt to approximately 45% wt of the composition of water; approximately 0.01% wt to approximately 2% wt sheer fragrance, wherein the sheer fragrance comprises approximately 10% wt to approximately 80% wt top notes based on the total weight of the sheer fragrance; optionally 0% wt to 5% wt odour neutralizing cationic surfactant; optionally 0% wt to approximately 0.43% wt of the composition of a corrosion inhibitor; optionally 0% wt to approximately 0.43% wt of the composition of a pH adjuster; optionally 0% wt to approximately 0.29% wt of the composition of a defoaming agent; and optionally 0% wt to approximately 0.43% wt of the composition of dye.

The disclosed air decontamination and malodour control compositions are packaged in a single use or multi-use aerosol can. The disclosed air decontamination and malodour control products comprise an aerosol can comprising approximately 70% wt to approximately 80% wt of the disclosed compositions and approximately 20% wt to approximately 30% wt of the propellant, both based on the total combined weight of the compositions and propellant. This concentration of the propellant helps to obtain the required concentration of DPG in air.

The disclosed air decontamination and malodour control compositions reduce the concentration of germs in the air while simultaneously reducing or suppressing the perception of malodour in the air. The germs may be in the air from oral or fecal transmission or from re-aerosolization from surfaces. Oral transmission may result from coughing, sneezing, or even just breathing while contagious. Fecal transmissions may result from flushing a toilet. Re-aerosolization may occur when a surface contaminated with germs is disturbed, whether by vacuuming or air disturbances from ceiling fans or opening doors or walking through a room. The disclosed air decontamination and malodour control compositions reduce the transmission of germs from the air to a surface. Stopping transmission helps prevent further infectious spread. As a result, the disclosed compositions reduce transmission of germs from air to surfaces.

For testing purposes, the air may be contained in an area less than or equal to 25 m³. One of ordinary skill in the art will recognize that air volume is only provided for comparative testing purposes and that the disclosed compositions may be used in smaller or larger areas without departing from the teachings herein. Between approximately 1.1 g/second to approximately 3.0 g/second, preferably approximately 1.5 g/second to approximately 2.5 g/second, of the aerosolized compositions disclosed above is introduced into the air for approximately 10 to approximately 60 seconds, preferably for approximately 10 to approximately 45 seconds, more preferably for approximately 10 to approximately 30 seconds. The air decontamination and malodour control compositions may be introduced into the air in one (1) spray or multiple sequential shorter sprays.

As shown in the Examples that follow, the disclosed compositions provide a 3 log₁₀ reduction of Staphylococcus aureus, Klebsiella pneumoniae, and Phi6 in 0.1 to 5 minutes. S. aureus is one of the more difficult bacteria to eradicate. K. pneumoniae is one of the easier bacteria to eradicate. Phi6 is a bacteriophage that acts as a testing surrogate for enveloped viruses, like SARS-CoV-2, corona and influenza viruses. The disclosed air decontamination and malodour control compositions may also be used to reduce the concentration of small non-enveloped viruses, as demonstrated in the examples that follow with the MS2 bacteriophage (Emesvirus zinderi), commonly called MS2. One of ordinary skill in the art will recognize that non-enveloped viruses are more difficult to inactivate than bacteria and enveloped viruses. As a result, the disclosed air decontamination and malodour control compositions take longer to inactivate MS2 than S. aureus, K. pneumoniae, and Phi6.

The disclosed products comprise an aerosol can comprising the disclosed compositions and a propellant. The aerosol can is loaded with the liquid composition and propellant to a pressure approximately equal to or slightly greater than the vapor pressure of the propellant. The product comprises approximately 70% wt to approximately 80% wt of the compositions disclosed above and approximately 20% wt to approximately 30% wt of propellant, both weight percentages based upon the combined weight of the composition and the propellant.

The propellant is a pressurized gas, preferably liquified petroleum gas (LPG). The propellant is not dimethyl ether (DME). DME is incompatible with many packaging components, such as plastic and rubber. LPG comprises a blend of propane and butane sufficient to produce at least 60 psig pressure, preferably from about 70 psig to about 80 psig. For example, the pressurized liquified petroleum gas may comprise approximately 40% wt to approximately 60% wt of the propane gas and approximately 40% wt to approximately 60% wt of the isobutane gas. One exemplary gas suitable for use with the teachings herein has 42.89% wt propane and 57.11% wt isobutane. This propellant is commercially available from multiple vendors as the A-70 hydrocarbon blend having a 70 psig pressure.

As shown in the examples that follow, germ kill is obtained when the concentration of DPG vapor in the air ranges from approximately 5 ppm to approximately 30 ppm, preferably from approximately 9 ppm to approximately 12 ppm. This concentration was accomplished by increasing the size of the stem orifice in the aerosol nozzle to produce larger spray particles, ranging from approximately Dv[50]=45 microns to approximately Dv[50]90 microns, preferably from approximately Dv[50]45 microns to approximately Dv[50]55 microns or approximately Dv[50]50 microns to approximately Dv[50]60 microns. Applicant believes that the initial supersaturation of the air with DPG obtained from the disclosed products provides the disclosed germ kill. Applicant further believes that hang time of the disclosed compositions provides the reduction and suppression of malodour in the air. More specifically, Applicant believes that the disclosed products provide a hang time lasting from approximately 0.1 minutes to approximately 120 minutes as demonstrated by the air analysis of DPG in the examples showing ongoing air concentrations for at least 20 minutes after spraying.

A 2-piece mechanical breakup (MB) nozzle with swirl chamber is connected to the aerosol can. The MB nozzle comprises two stem orifices, each of which have a diameter ranging from approximately 0.036 inches [0.91 mm] to approximately 0.044 inches [1.12 mm]. The size of each of the two stem orifices in the disclosed air decontamination and malodour control product increases the discharge rate of the air decontamination and malodour control composition. The size of the stem orifices also increases the particle size of the resulting spray. The 2-piece MB nozzle is commercially available.

By manipulating particle size via the aerosol package configuration, preferential sizes may be generated. The ability to generate preferential particle sizes enhances the creation of DPG in the vapor state. The resulting spray has a particle size ranging from approximately Dv[50]=45 microns to approximately Dv[50]=90 microns, preferably from approximately Dv[50]=50 microns to approximately Dv[50]=60 microns or from approximately Dv[50]=45 microns to approximately Dv[50]=55 microns. One of ordinary skill in the art will recognize that Dv[50] is the maximum particle diameter below which 50% of the sample volume exists. There are a wide range of commercially available particle size measuring apparati available, including but not limited to laser diffraction, dynamic light scattering, electrophoretic light scattering, automated imaging, sedimentation, electrozone sensing, or sieving. Laser diffraction, automated imaging, electrozone sensing, and sieving are preferred techniques for the disclosed particle sizes, with laser diffraction particularly preferred.

The resulting spray has a volume moment mean ranging from Dv[4,3]=55 microns to Dv[4,3]=75 microns on average as measured using a laser diffractometer, preferably from Dv[4,3]=60 microns to Dv[4,3]=70 microns. The volume moment mean reflects the size of most of the particles in the sample.

As shown in the examples that follow, testing was performed using two or four stem orifices. While four stem orifices help to increase the flow rate of the disclosed air decontamination and malodour control composition, the particle size in the resulting spray was inconsistent and irregular. The size of the two stem orifices in the disclosed air decontamination and malodour control product produce a less turbulent flow of the needed volume and spray size of air decontamination and malodour control composition as compared to four stem orifices.

Applicant was surprised by the required stem orifice and spray particle sizes. One of ordinary skill in the art would not expect that these parameters would successfully produce vaporized glycol. One of ordinary skill in the art would expect that better vaporization would occur using smaller particle spray. The increased discharge rate resulting from the claimed stem orifice size floods the air with vaporized material, producing between approximately 5 ppm to approximately 30 ppm DPG vapors in the air, preferably between approximately 9 ppm to approximately 12 ppm DPG vapors. This air saturation level creates an environment where the glycol partitions onto the germ surface. The DPG creates a liquid film on the surface of any bacterium in the air, resulting in osmotic imbalance of the bacterium and subsequent inactivation. Applicant believes that the DPG interferes with the virus-host interaction by impairing viral receptor-binding domain required to attach to host cell, which prevents the virus particles from replicating.

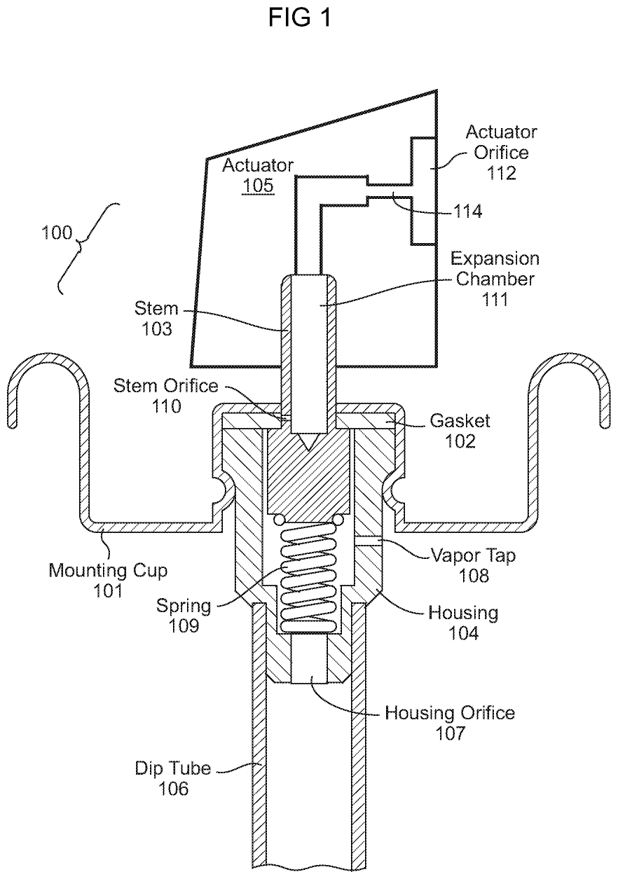

FIG. 1 is a diagram of an exemplary 2-piece MB nozzle aerosol valve assembly 100 that may be used to dispense the disclosed air decontamination and malodour control compositions. The valve assembly 100 is connected to the mounting cup 101. The mounting cup 101 is crimped to the aerosol can (not shown) in an air-tight manner. A gasket 102 seals the junction between the top of the valve assembly housing 104, mounting cup 101, and stem 103. The stem 103 extends from the valve assembly housing 104 through the gasket 102 and mounting cup 101 to the actuator 105. The valve assembly housing 104 includes a housing orifice 107, vapor tap 108, and spring 109. The spring 109 holds the valve assembly housing 104 in the closed position. The stem 103 includes a stem orifice 110 and an expansion chamber 111. The single stem orifice 110 in FIG. 1 is provided for illustration purposes only. The two stem orifices 110 in the disclosed product are located on opposite sides of the stem 103. However, the location of the two stem orifices 110 should not have an impact on the resulting particle spray rate and size. The actuator 105 includes a swirl chamber 114 in fluid connection with an actuator orifice 112. One of ordinary skill in the art will recognize that actuators 105 that include a swirl chamber 114 are known as mechanical break up actuators 105. A mechanical break up insert (not shown) in the swirl chamber 114 generates the swirl, producing the 2-piece mechanical breakup (MB) nozzle aerosol valve assembly 100 with swirl chamber 114 or break up bar. The actuator 105 in FIG. 1 depicts a right angle from the stem 103 to the actuator orifice 112. One of ordinary skill in the art will recognize that a straight pathway may also be used without departing from the teachings herein. A dip tube 106 is connected to the bottom of the valve assembly housing 104.

When the actuator 105 is depressed, the stem 103 moves downward, which opens the seal between the gasket 102 and the stem orifice 110. The propellent forces the disclosed air decontamination and malodour control compositions into the dip tube 106 and through the housing orifice 107 into the valve assembly housing 104. Propellant introduced into the valve assembly housing 104 through the vapor tap 108 mixes with the disclosed air decontamination and malodour control composition inside the valve assembly housing 104. The propellant both dries the air decontamination and malodour control composition as well as begins formation of aerosol droplets and subsequent glycol vapors. The generation of glycol vapors is absolutely critical in the denaturation of the airborne microbiological species. Applicant has found that the propellant must have at least 60 psig pressure in order to vaporize the disclosed air decontamination and malodour control compositions. The air decontamination and malodour control composition/propellant blend moves from the valve assembly housing 104 through the stem orifice 110 into the expansion chamber 111. From the expansion chamber 111, the composition moves through the actuator 105 to the swirl chamber 114 and out the actuator orifice 112 as an aerosolized spray.

Numerous swirl chambers 114 are commercially available. See, e.g., U.S. Pat. No. 3,583,642 to SC Johnson & Son, Inc., the contents of which are incorporated herein in its entirety by reference. The swirl chamber 114 is one factor in the production of aerosol particles of the desired size. Initial R&D tests conducted without a swirl chamber 114 resulted in visible spray on the floor below the nozzle. One of ordinary skill in the art will recognize that a pin orifice may also produce a suitably sized aerosol particle.

The size of the actuator orifice 112 combined with the concentration of the propellant in the disclosed air decontamination and malodour control products provides projection of the spray. The aerosol bloom travels approximately 7 feet (2.1 meters) to approximately 9 feet (2.7 m) from the actuator orifice. This combination permits the larger sized aerosol time to shrink due to evaporation. This combination also permits the aerosol to cover more geography than standard fine mist sprays. The diameter of the actuator orifice ranges from approximately 0.016 inches (0.41 mm) to approximately 0.022 inches (0.56 mm), preferably from approximately 0.017 inches (0.43 mm) to approximately 0.021 inches (0.53 mm), and more preferably from approximately 0.018 inches (0.46 mm) to approximately 0.020 inches (0.51 mm). Alternatively, the diameter of the actuator orifice ranges from 0.016 inches (0.41 mm) to 0.022 inches (0.56 mm), preferably from 0.017 inches (0.43 mm) to 0.021 inches (0.53 mm), and more preferably from 0.018 inches (0.46 mm) to 0.020 inches (0.51 mm).

The size of the vapor tap 108 is another factor that helps determine the size of the aerosol particles. Decreasing the size of the vapor tap 108 lowers the ratio of the propellant to air decontamination and malodour control composition and reduces the amount of composition retention in the can. But decreasing the size of the vapor tap 108 also increases the aerosol particle size, which may prevent aerosolization of the composition due to the low vapor pressure of DPG. In other words, as shown in some of the examples that follow, too large an aerosol particle size of the disclosed air decontamination and malodour control compositions results in liquid being visible on the floor below the nozzle. Liquid on the floor does not provide effective air sanitization. As discussed above, increasing the pressure of the propellant permits the larger sized aerosol spray the ability to shrink over distance and time. As a result, the disclosed air decontamination and malodour control products have a pressure of 60 psig or higher. This difference is most evident in comparison of Examples D and N.

The size of the housing orifice 107 also contributes to the size of the aerosol particles. Decreasing the size of the housing orifice 107 decreases the aerosol particle size.

The size of the stem orifice 110 also contributes to the size of the aerosol particles. Decreasing the size of the stem orifice 110 decreases the aerosol particle size.

The 2-piece MB nozzle aerosol valve assembly 100 comprises an actuator orifice 112 having a diameter ranging from approximately 16 μm to approximately 22 μm, a vapor tap 108 having a diameter ranging from approximately 0.016 inches [0.41 mm] to approximately 0.025 inches [0.64 mm], and two stem orifices 110, each having a diameter ranging from approximately 0.036 inches [0.91 mm] to approximately 0.044 inches [1.12 mm].

Alternatively, the 2-piece MB nozzle aerosol valve assembly 100 comprises an actuator orifice 112 having a diameter ranging from approximately 17 μm to approximately 21 μm, a vapor tap 108 having a diameter ranging from approximately 0.017 inches [0.43 mm] to approximately 0.024 inches [0.61 mm], and two stem orifices 110, each having a diameter ranging from approximately 0.037 inches [0.94 mm] to approximately 0.043 inches [1.09 mm].

In another alternative, the 2-piece MB nozzle aerosol valve assembly 100 comprises an actuator orifice 112 having a diameter ranging from approximately 18 μm to approximately 20 m, a vapor tap 108 having a diameter ranging from approximately 0.018 inches [0.43 mm] to approximately 0.023 inches [0.61 mm], and two stem orifices 110, each having a diameter ranging from approximately 0.038 inches [0.97 mm] to approximately 0.042 inches [1.07 mm].

In another alternative, the 2-piece MB nozzle aerosol valve assembly 100 comprises an actuator orifice 112 having a diameter ranging from approximately 18 μm to approximately 20 m, a vapor tap 108 having a diameter ranging from approximately 0.019 inches [0.48 mm] to approximately 0.022 inches [0.56 mm], and two stem orifices 110, each having a diameter ranging from approximately 0.038 inches [0.97 mm] to approximately 0.042 inches [1.07 mm].

As shown in the Examples that follow, the combination of the disclosed air decontamination and malodour control compositions and the 2-piece MB nozzle aerosol valve assembly 100 sanitizes air in 0.1 to 5 minutes. The actuator 105 is depressed for approximately 10 seconds to approximately 60 seconds to introduce between approximately 1.1 g/second to approximately 2 g/second of the air decontamination and malodour control composition into a volume of air occupying less than or equal to 25 m³. The actuator 105 may be depressed manually. Alternatively, the actuator 105 may automatically remain depressed for the desired dispensing duration. For example, the valve assembly 100 may include a solenoid switch as disclosed in PCT Publication Nos. WO2007/045826, WO2007/045827, and WO2007/045828, all to Reckitt Benckiser (UK) Ltd., the contents of which are incorporated herein by reference in their entireties. More particularly, the valve assembly 100 may comprise a moveable magnetic stem 103 surrounded by copper windings (not shown), with an iron frame (not shown) surrounding the copper windings. Electric current applied to the copper windings moves the magnetic stem 103 to either the open or closed position.

The following example below illustrates exemplary embodiments of the invention. It is to be understood that these examples are provided by way of illustration only and that further embodiments may be produced in accordance with the teachings of the present invention.

### EXAMPLES

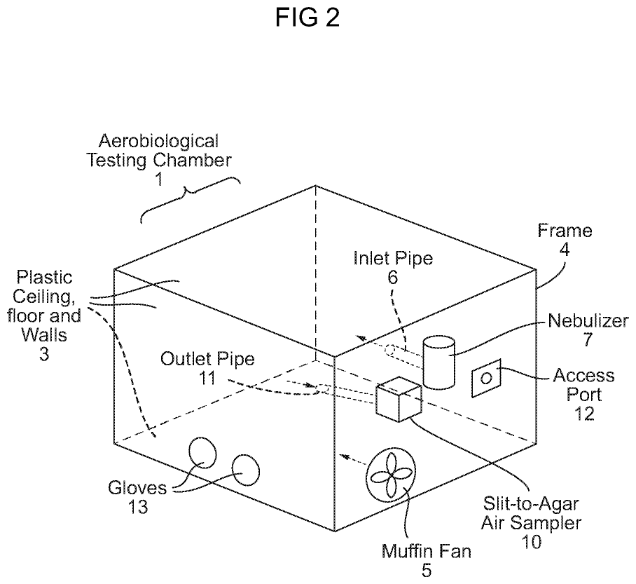

The following examples were performed in the aerobiological testing chamber 1 as shown in FIG. 2 (which is not to scale). The aerobiological testing chamber 1 has a volume of approximately 24 m³ to approximately 25 m³. The chamber 1 is located inside a clean room (not shown) with negative pressure and control access. The ceiling, floor, and walls of the chamber 1 are made of a wipeable white corrugated plastic sheet 3 affixed to the frame 4 of the chamber 1 to maintain an airtight seal preventing air exchange between the chamber 1 and the clean room. While other colors may be used without departing from the teachings herein, the white corrugated plastic sheets provide better visibility for any residual product stains. The wipeable corrugated plastic sheet 3 used in the examples that follow was 0.0157 inches (0.4 cm) thick and sold at Home Depot under the Coroplast™ brand by Coroplast Fritz Müller GmbH & Co. The walls should be grounded to dissipate any static electricity that may accumulate. A static electricity discharge wire (not shown) is provided to help discharge any static electricity buildup. The chamber 1 may further include clear plastic windows, sealable access doors, and inlets and outlets for the HEPA filtration system. More detailed depictions of some exemplary chambers 1 may be found in Sattar et al., Spread of viral infections by aerosols, Crit Rev Environ Control, 1987, 17:89-131; Sattar et al., Decontamination and malodour control of indoor air to reduce the risk of airborne infections: Studies on survival and inactivation of airborne pathogens using an aerobiology chamber, American Journal of Infection Control, 44 (2016) e177-e182, the contents of both being incorporated herein by reference in their entireties.

A muffin fan 5, also known as an axial flow fan, is placed on the floor inside the chamber 1 directly underneath the 3.8 cm diameter inlet pipe 6 to a 6-jet nebulizer 7. The 6-jet collision nebulizer 7 generates microbial aerosols in the respirable range of 0.5-5.0 μm. The nebulizer 7 used in the following examples was Model MRE CN24/25 purchased from CH Technologies of Westwood, NJ, US. The nebulizer 7 was connected to a cylinder of extra-dry compressed air with pressure regulator and backflow preventer (neither shown). The fan 5 used was a Nidec Alpha V, TA300, Model A31022-20, Part number 933314 3.0 inch/7.62 m diameter, output 30 CFM supplied by Nidec Corp of Braintree, MA, US. A data recorder (not shown) records the chamber's relative humidity and air temperature. The following examples used the RTR-503L model of wireless data loggers from CAS Data Loggers of Chesterland, OH, US. A magnehelic (not shown) records the pressure differential between the inside and the outside of the chamber 1. The following examples used a magnehelic purchased from ITM Instrument Inc. of Ontario, Canada.

The air in the chamber 1 is sampled at the rate of 1 ft³ (28.3 L)/minute using an externally placed slit-to-agar air sampler with a built-in vacuum pump 10. The sampler 10 used in the examples was purchased from PinPoint Scientific of Bridgend, Wales. The air exiting the sampler 10 is captured in a HEPA filter incorporated in the device 10 or discharged directly into the facility's HEPA-filtered exhaust system (not shown). The sampler 10 draws air samples from the center of the chamber 1 through a 5.0 cm diameter outlet pipe 11.

As discussed by Zargar et al. at page S136, the fan 5 provides uniform distribution of the aerosolized particles in the air inside the chamber 1 when placed at a 450 angle at the bottom of one side of the chamber 1 and operated at 2800 RPM. Mathematical modeling and simulation of bacterial distribution in an aerobiology chamber using computational fluid dynamics, American Journal of Infection Control 44 (2016) S127-S137, incorporated herein in its entirety herein by reference. Zargar et al. further disclose that a 5-minute post-nebulization time is sufficient to distribute introduced bacteria aerosols uniformly throughout the chamber. Id. Zargar et al. further disclose that collection of air samples from the center of the chamber 1 was sufficient to provide a representative profile of the concentration of the airborne bacteria present within the chamber 1. Id.

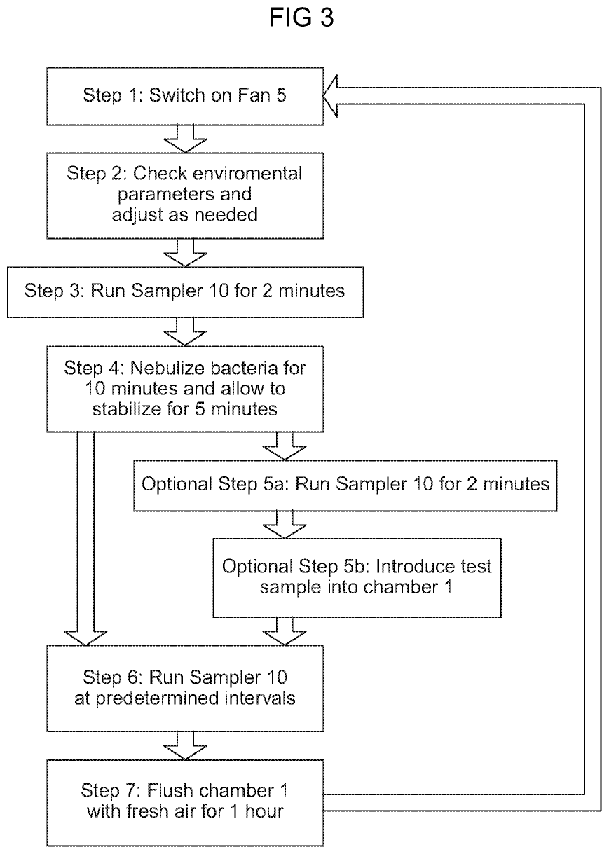

FIG. 3 is a flow chart of the steps performed in the following examples. In step 1, the fan 5 is turned on to start air circulation within the chamber 1. In step 2, the environmental parameters inside the chamber 1 are reviewed and adjusted as needed. Step 3 provides for activation of the slit-to-agar sampler 10 for 2 minutes in order to obtain a sample of any background contamination in the chamber 1. After the sampler 10 is turned off, the bacteria, virus, or phage to be tested is introduced into the chamber 1 via the nebulizer 7 and inlet pipe 6. In this step 4, the nebulizer 7 is run for 10 minutes. The nebulizer 7 is then turned off and the bacteria, virus, or phage allowed to stabilize inside the chamber 1 for 5 minutes. Step 4 should produce a concentration of bacteria, virus, or phage between a minimum of 4.2 log₁₀/m³ to a maximum of 5.0 log₁₀ CFU/m³ in chamber 1.

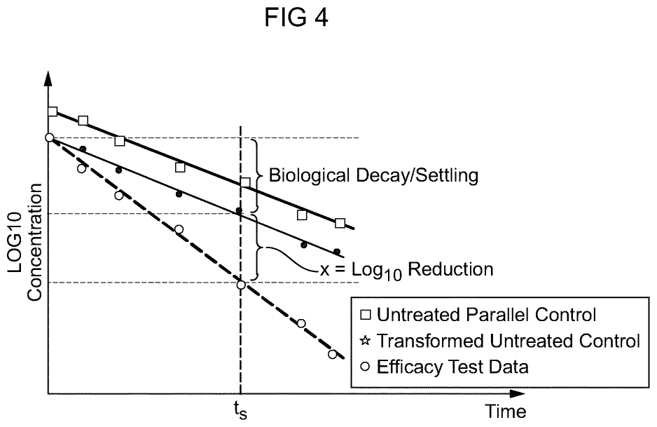

Any meaningful assessment of air decontamination requires that the aerosolized challenge microbes remain viable in the air long enough to allow for proper differentiation between its biological decay or physical fallout and inactivation or removal by the technology being assessed. The test microbes (i.e., bacteria and bacteriophages) were aerosolized into the chamber 1 in Step 4. Steps 5a and 5b are initially skipped to provide the comparative baseline reading of the biological decay or physical fallout. In step 6, samples are collected using a slit-to-agar sampler 10 at predetermined intervals over a specified time frame, for example every 2 minutes over an 8-hour period. The culture plates were incubated at 36 °C±1 °C, the colony forming units (CFU) or plaque forming units (PFU) recorded, and the data analyzed to determine the rate of biologic decay. The results are shown in FIG. 4. The chamber 1 was then flushed with fresh air for one hour in Step 7 to decontaminate it and the process started over at Step 1.

Steps 1 to 4 are repeated for different product samples. In Step 5a, the slit-to-agar sampler 10 is run for 2 minutes to determine the initial concentration of the challenge microbes. In Step 5b, the decontamination and malodour control product to be tested is introduced into the chamber 1 through an access port 12 in the wall 2 of the chamber 1.

Alternatively, the decontamination and malodour control sample may be placed in the chamber 1 prior to step 1 and accessed and activated using gloves 13 affixed to the wall 2 of the chamber 1 in step 5b. In step 6, samples are collected using a slit-to-agar sampler 10 at predetermined intervals over a specified time frame, for example every 2 minutes over an 8-hour period. The interval and time period for the determination of the biological decay and physical fallout should match the interval and time period used to determine inactivation using the test product. The culture plates were incubated at 36 °C±1 °C, the colony forming units (CFU) or plaque forming units (PFU) recorded, and the data analyzed to determine the rate of inactivation. The results are shown in FIG. 4. The chamber 1 was then flushed with fresh air for one hour in Step 7 to decontaminate it and the process started over with a new sample at Step 1.

FIG. 4 is an exemplary graph of the log₁₀ concentration of the tested microorganism versus time. The thick solid black line shows the rate of biological decay of the challenge bacterial species. The dotted black line shows the rate of inactivation of the challenge bacterial species by the test product. Since the initial titers of the two experiments may differ, the data from the biological decay is transformed so that its initial titer becomes equal to the initial titer for the inactivation test. This transformation step is easily accomplished by one of ordinary skill in the art. The modified biological decay is shown by the thin black line parallel to the thick black line. Successful disinfection results are obtained when the log₁₀ reduction from natural or biological decay (thin black line) to inactivation (dotted line) is equal or greater than 3 log₁₀ after a 5 minute or less contact time tₛ.

The lyophilized test microbes are obtained from a reputable source, such as the American Type Culture Collection (ATCC). The microbes are isolated using standard techniques. The bacteria are cultured to provide approximately 1.6×10⁴ CFU/m³ to approximately 1.0×10⁵ CFU/m³. The nebulization fluid is prepared by adding 50 μL of the cultured bacteria, 0.75 mL Bovine Serum Albumin (BSA), 1.05 mL yeast extract, 3.0 mL mucin, and 10 μL of Antifoam A (from Sigma-Aldrich, Cat A-5633) to 10.14 mL of Dulbecco's Phosphate-Buffered Saline (PBS).

The compositions in the following examples were prepared using the ingredients identified in Table A:

TABLE A

| Abbreviation | CAS | Description |
|---|---|---|
| H₂O | 7732-18-5 | Deionized Water |
| CI | 26038-87-9 | Corrosion inhibitor, e.g., MEA Borate and MIPA Borate sold |
| | and | under the tradename Crodacor ™ BE by Croda |
| | 26038-90-4 | |
| MEA | 141-43-5 | Monoethanolamine |
| EtOH | 67-17-5 | Denatured Ethanol, e.g., SDA 40B sold by MGP |
| DPG | 25265-71-8 | DiPropylene Glycol, e.g., Regular Grade Dipropylene Glycol |
| | | sold by Dow |
| TEG | 112-27-6 | TriEthylene Glycol |
| OFX | 68937-54-2 | PEG-12 Dimethicone, e.g., Xiameter OFX-0193 Fluid sold by |
| | | Dow |
| SME | 61791-34-2 | Soyethyl Morpholinium Ethosulfate, e.g., Forestall-LQ-(HM) |
| | | sold by Croda at 35% w/w purity in water, alcohol and/or |
| | | polypropylene glycol |
| Quat | 68989-01-5 | Alkyl dimethyl benzyl ammonium saccharinate, e.g., Oxynide |
| | | 3300 sold by Stepan |
| F | Various | Fragrance |
| Prop NP-46 | | Propellant blend of 25.86% w/w propane and 74.14% w/w N- |
| | | butane that has a vapor pressure of 46 psig |
| Prop | | Propellant blend of 21.02% w/w propane and 59.32% w/w |
| 152a/AB-46 | | isobutane that has a vapor pressure of 66 psig |
| Prop A-70 | | Propellant blend of 42.89% w/w propane and 57.11% w/w |
| | | isobutane that has a vapor pressure of 70 psig |

The compositions in the following examples were tested using the aerosol nozzles identified in Table B, all of which utilized 2-piece Mechanical Break-up actuators (“2-piece NIB”) with swirl chambers.

TABLE B

| | V1 | V2 | V3 | V4 | V5 |
|---|---|---|---|---|---|
| Valve Vapor Tap in | 0.018 | 0.016 | 0.032 | 0.016 | 0.020 |
| inches [mm] | [0.46] | [0.41] | [0.82] | [0.41] | [0.51] |
| Valve Housing Orifice in | 0.025 | 0.025 | 0.032 | 0.025 | 0.025 |
| inches [mm] | [0.64] | [0.64] | [0.81] | [0.64] | [0.64] |
| Valve Stem Orifice, | 4 × 0.024 | 2 × 0.025 | 4 × 0.024 | 2 × 0.025 | 2 × 0.04 |
| number of orifices and | [0.61] | [0.64] | [0.61] | [0.64] | [1.02] |
| diameter of each orifice | | | | | |
| in inches [mm] | | | | | |
| Actuator Exit Orifice in | 0.025 | 0.020 | 0.025 | 0.020 | 0.019 |
| inches [mm] | [0.64] | [0.51] | [0.64] | [0.51] | [0.48] |
| Actuator Lan Length in | 0.031 | 0.010 | 0.025 | 0.040 | 0.020 |
| inches [mm] | [0.79] | [0.25] | [0.64] | [1.02] | [0.51] |

### Example 1: S. aureus

R&D samples of the following compositions were tested against Staphylococcus aureus (ATCC 6538).

The compositions and aerosol nozzles used in the testing are provided in Table 1 (all amounts in 00 wt based on the total combined weight of the compositions and propellant):

TABLE 1

| | | | EtOH | | | | | | | |
|---|---|---|---|---|---|---|---|---|---|---|
| | | | (190 | | | | SME | | | |
| Ref | H₂O | DPG | Proof) | CI | MEA | OFX | (35%) | F | Prop | Valve |
| A | 39.45 | 25 | 10 | 0.1 | 0.1 | 0.05 | 0.2 | 0.1 | 25 152a/ | V3 |
| | | | | | | | (0.07) | | AB-46 | |
| B | 26.6 | 7.5 | 40 | 0.2 | 0.2 | 0.075 | 0.3 | 0.125 | 25 | V1 |
| | | | | | | | (0.1) | | NP-46 | |
| C | 24.1 | 10 | 40 | 0.2 | 0.2 | 0.075 | 0.3 | 0.125 | 25 | V1 |
| | | | | | | | (0.1) | | NP-46 | |
| D | 19.1 | 15 | 40 | 0.2 | 0.2 | 0.075 | 0.3 | 0.125 | 25 | V1 |
| | | | | | | | (0.1) | | NP-46 | |
| E | 19.45 | 25 | 30 | 0.1 | 0.1 | 0.05 | 0.2 | 0.1 | 25 | V4 |
| | | | | | | | (0.07) | | NP-46 | |
| F | 19.45 | 25 | 30 | 0.1 | 0.1 | 0.05 | 0.2 | 0.1 | 25 152a/ | V1 |
| | | | | | | | (0.07) | | AB-46 | |
| G | 19.45 | 25 | 30 | 0.1 | 0.1 | 0.05 | 0.2 | 0.1 | 25 152a/ | V3 |
| | | | | | | | (0.07) | | AB-46 | |
| H | 19.45 | 25 | 30 | 0.1 | 0.1 | 0.05 | 0.2 | 0.1 | 25 152a/ | V4 |
| | | | | | | | (0.07) | | AB-46 | |
| I | 19.45 | 25 | 30 | 0.1 | 0.1 | 0.05 | 0.2 | 0.1 | 25 | V4 |
| | | | | | | | (0.07) | | A-70 | |
| J | 9.45 | 35 | 30 | 0.1 | 0.1 | 0.05 | 0.2 | 0.1 | 25 | V3 |
| | | | | | | | (0.07) | | A-70 | |
| K | 9.45 | 35 | 30 | 0.1 | 0.1 | 0.05 | 0.2 | 0.1 | 25 | V4 |
| | | | | | | | (0.07) | | A-70 | |
| L | 19.53 | 25 | 30 | 0.07 | 0.1 | 0 | 0.2 | 0.1 | 25 | V2 |
| | | | | | | | (0.07) | | A-70 | |
| M | 19.53 | 25 | 30 | 0.07 | 0.1 | 0 | 0.2 | 0.1 | 25 | V4 |
| | | | | | | | (0.07) | | A-70 | |
| N | 25.46 | 14 | 35 | 0.07 | 0.1 | 0 | 0.2 | 0.17 | 25 | V5 |
| | | | | | | | (0.07) | | A-70 | |

The particle size generated by Formula N was measured using a Malvern laser diffractometer with a 300 mm lens 6 inches from the beam. The average Dv(50) of 3 separate measurements was 54.83 microns. The average volume moment mean Dv[4,3] of 3 separate measurements was 63.88 microns.

Spray efficacy was demonstrated by obtaining a 3 log₁₀ reduction of Staphylococcus aureus. When available, the spray time, temperature, percent relative humidity (0% RH), and results are provided in Table 2:

TABLE 2

| | | | | | | (a) Log₁₀ | (b) Time | | Spray |
|---|---|---|---|---|---|---|---|---|---|
| | Spray | Wt. Comp | Wt. DPG | | | reduction/ | (minutes)/ | | Visible |
| | Time (In | Sprayed | Sprayed | Temp | % | time | 3-LR | Achieve | on |
| Ref | Seconds) | (g) | (g) | (°C) | RH | (minutes) | achieved? | Sanitization? | Floor? |
| A | N/A | N/A | N/A | N/A | N/A | N/A | N/A/No | No | N/A |
| B | 60 | 86.78 | 6.5 | 22.5 | 49 | 4.30/120 | 55/Yes | No | No |
| C | N/A | N/A | N/A | N/A | N/A | N/A | N/A/No | No | N/A |
| D | 10 | 11.24 | 1.7 | 23 | 51 | 0.12/105 | 120/No | No | Yes |
| D | 60 | 51.19 | 7.7 | 22.7 | 54 | 0.40/120 | 120/No | No | Yes |
| E | N/A | N/A | N/A | N/A | N/A | N/A | N/A/No | No | N/A |
| F | 15 | 13.53 | 3.4 | 23 | 51 | 3.31/120 | 65/Yes | No | No |
| F | 15 | 11.53 | 2.9 | 23 | 51 | 0.8/120 | 120/No | No | No |
| F | 15 | 12.78 | 3.2 | 22 | 50 | 1.1/120 | 120/No | No | No |
| F | 15 | 9.65 | 2.4 | 23 | 52 | 1.4/120 | 120/No | No | No |
| F | 30 | 17.29 | 4.3 | 22.5 | 47 | 4.21/120 | 3.70/Yes | Yes | No |
| F | 60 | 60.56 | 15.1 | 23 | 45 | 4.34/120 | 4.37/Yes | Yes | No |
| G | N/A | N/A | N/A | N/A | N/A | N/A | N/A/Yes | Yes | N/A |
| H | N/A | N/A | N/A | N/A | N/A | N/A | N/A/Yes | Yes | N/A |
| I | 15 | 12.81 | 3.2 | 22 | 55 | 1.66/120 | 120/No | No | No |
| I | 20 | 18.77 | 4.7 | 23 | 49 | 2.48/120 | 10/Yes | No | No |
| I | 25 | 20.70 | 5.2 | 21 | 53 | 3.97/120 | 5.17/Yes | No | No |
| I | 25 | 19.92 | 5.0 | 25.4 | 53 | 2.86/120 | 5.75/Yes | No | No |
| I | 30 | 32.77 | 8.2 | 22.5 | 53 | 4.11/120 | 3.91/Yes | Yes | No |
| I | 30 | 22.28 | 5.6 | 24.4 | 51.5 | 3.86/120 | 6.32/Yes | No | No |
| I | 30 | 36.68 | 9.2 | 24.4 | 51.5 | 4.06/120 | 3.91/Yes | Yes | No |
| J | 20 | 18.29 | 6.4 | 25.7 | 54 | 1.70/120 | 120/No | No | No |
| K | N/A | N/A | N/A | N/A | N/A | N/A | N/A/No | No | N/A |
| L | 20 | 26.19 | 6.5 | 24.5 | 55 | 1.5/5 | N/A/No | No* | |
| | | | | | | 2.5/105 | | | |
| L | 30 | 37.67 | 9.4 | 24.5 | 54 | 4.50/120 | 4.12/Yes | Yes | No |
| M | N/A | N/A | N/A | N/A | N/A | N/A | N/A/Yes | Yes | N/A |
| N | 30 | 30.21** | 4.2** | 23.9** | 49** | 3/2.86** | 2.86** | Yes | No |

N/A—this data is not currently available
*Small droplets below the can, but not on floor - may be condensation
**average of 3 runs

Table 2 provides both (a) the time it takes to obtain the specified log₁₀ reduction of Staphylococcus aureus and (b) the time it takes to obtain a 3 log₁₀ reduction in Staphylococcus aureus, if indeed a 3 log₁₀ reduction in Staphylococcus aureus was obtained. The composition is considered a successful air disinfectant when it obtains a 3 log₁₀ reduction in Staphylococcus aureus in 5 minutes or less.

One of ordinary skill in the art will recognize that microbiological test results are not as consistent as chemical test results. Microbiological test results can vary from test to test, even when all other parameters remain the same. Higher variation in microbiological test results is also to be expected in R&D test environments. One of ordinary skill in the art will further recognize that the concentration of bacteria in the air over time may fluctuate. Additionally, the air being sampled in real time may contain residual bacteria when the concentration of the product is sub-optimal/not efficient enough to quickly kill all the bacteria. In other words, multiple factors may have contributed to the results obtained by Composition I, which was able to achieve a 3 log₁₀ reduction in S. aureus in 5.75 minutes after a 25 second spray, but the reduction decreased to 2.86 log₁₀ after 120 minutes.

As can be seen, the combination of the low concentration of DPG and the low pressure of Compositions B, C, and D did not produce sufficient vapor pressure to achieve sanitization, notwithstanding the higher concentration of ethanol. Composition E demonstrates that an increase in the concentration of DPG is not sufficient to overcome the limitations of the low-pressure NP-46 propellant. Initially, optimal results were obtained from aerosol compositions containing a ratio of dipropylene glycol:water:alcohol of approximately 1.25:1:1.5. Further testing demonstrated that the ratio of dipropylene glycol:water:alcohol preferably ranges from approximately 1:2:2.3 to 1:1.8:2.5 for aerosol applications.

The approximate 2:1 alcohol:water composition D produced visible spray on the floor. Applicant believes the propellant may cause the higher concentration of ethanol to evaporate more quickly. No spray was visible on the floor for the aerosol compositions containing a ratio of alcohol:water of approximately ranging from 1.3:1 to 1.5:1.

The 30 second spray of formula F, I, and N and the 60 second spray of formula F successfully achieved air disinfectant germ kill. The 15 second spray results for formula F demonstrate marginal effectiveness that may occasionally generate 3 log₁₀ reduction. In contrast, repeated testing of formula I and N at 30 seconds produces more consistent effectiveness results. Formula N was also tested against K. pneumoniae (ATCC 4352). ≥3 log₁₀ reduction was achieved in 1.17 minutes in the presence of a soil load. This result is expected because inactivation of K. pneumoniae is easier than inactivation of S. aureus.

Table 2 further demonstrates that increasing the spray time (e.g., 60 seconds for Formula B and D) or the percentage of DPG in the composition (e.g., 35% in Compositions J and K) were not alone sufficient to provide suitable microbiocidal outcome. Compositions J and K were too viscous to move DPG into the vapor phase, even with 70 psig pressure.

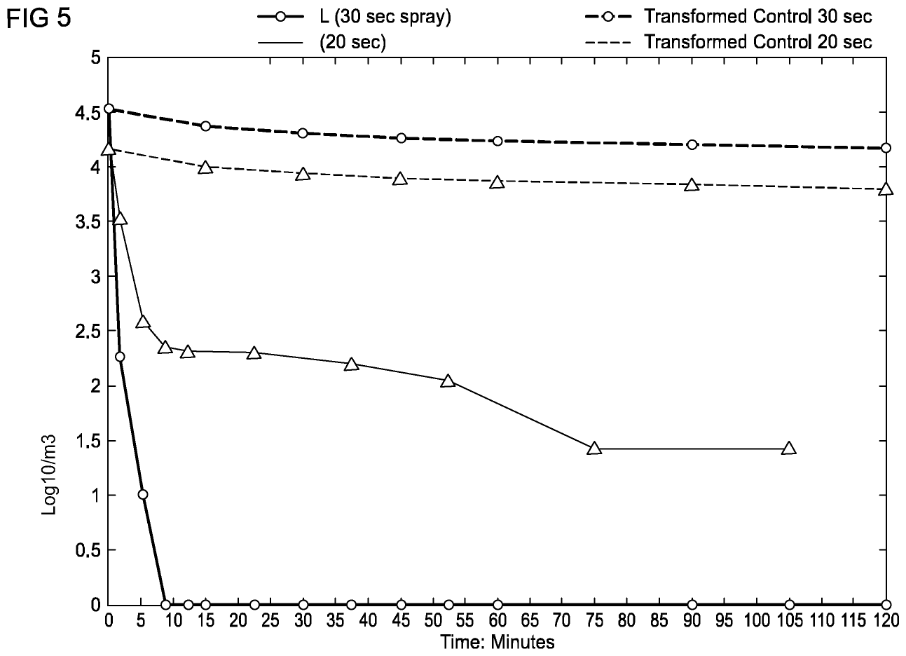

FIG. 5 is a graph of the log₁₀ concentration of S. aureus versus time for Composition L sprayed for 20 seconds (unbolded line with triangles) and 30 seconds (bolded line with circles). The corresponding dotted lines show the rate of biological decay of the challenge bacterial species. As can be seen, both the 20 and 30 second spray times provide fast reduction in S. aureus concentrations, but only the 30 second spray time provides the 3 log₁₀ reduction in less than 5 minutes.

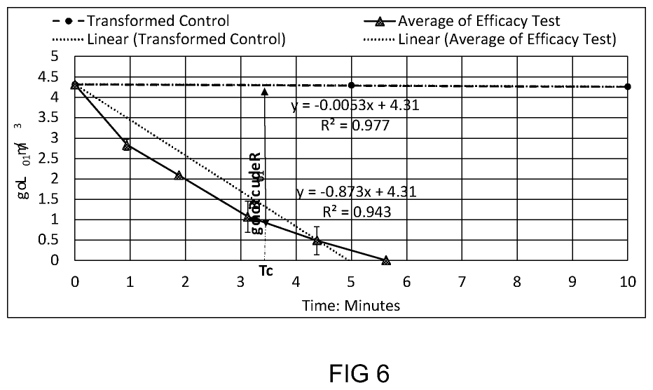

FIG. 6 is a graph of the average log₁₀ concentration of S. aureus versus time for three different lots of Composition N at a 30 second spray time, each lot tested in triplicate (so the average of 9 test results). As can be seen, Composition N obtains a 3 log₁₀ reduction in less than 4 minutes, notwithstanding having a lower concentration of DPG than Composition L. These results demonstrate that concentration of DPG in the air is more important than concentration of DPG in the composition.

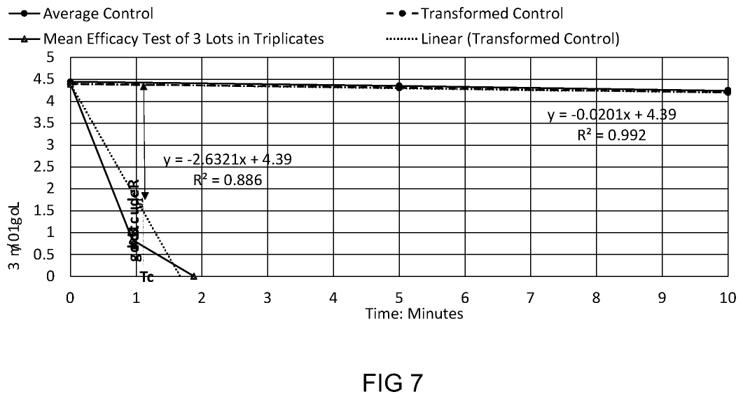

FIG. 7 is a graph of the average log₁₀ concentration of K. pneumoniae versus time for three different lots of Composition N at a 30 second spray time, each lot tested in triplicate (so the average of 9 test results). As can be seen, Composition N obtains a 3 log 10 reduction in less than 1 minutes.

One of ordinary skill in the art will recognize that a 20- to 30-second spray time is longer than an average consumer expects to depress a spray button. Applicant expects similar efficacy results after multiple but consecutive shorter spray times. For example, ten (10) consecutive 2- or 3-second sprays or four (4) to six (6) consecutive 5-second sprays, with a <1- to 2-second pause between sprays, are expected to produce similar results.

### Example 2: Phi6 and MS2

Spray efficacy was demonstrated by obtaining a 3 log₁₀ reduction of Phi6 or MS2 after a 30 second spray time. The spray time, temperature, percent relative humidity (% RH), and results are provided in Table 3:

TABLE 3

| | | | | | | Time | |
|---|---|---|---|---|---|---|---|
| | | Wt. | Wt. DPG | | | (minutes) to | |
| | | Composition | Sprayed | Temp | % | achieve | Spray Visible |
| Ref | Phage | Sprayed (g) | (g) | (°C) | RH | 3-LR | on Floor? |
| J | Phi6 | 29.69 | 7.4 | 24 | 52 | 4.07 | No |
| J | Phi6 | 32.81 | 8.2 | 23.5 | 49.5 | 3.85 | No |
| J | MS2 | 33.28 | 8.3 | 23.4 | 54 | 14.09 | No |
| J | MS2 | 26.77 | 6.7 | 22.8 | 49 | 13.57 | No |
| N | MS2 | 28.27 | 4.0 | 22.5 | 50 | 9.80 | No |
| N | MS2 | 30.03 | 4.2 | 22.5 | 50 | 11.3 | No |

As discussed above, Table 3 confirms that non-enveloped viruses like MS2 are more difficult to inactivate than bacteria and enveloped viruses like Phi6. That notwithstanding, a 30-second spray of the Composition J and N is still capable of providing a 3 log₁₀ reduction in concentration.

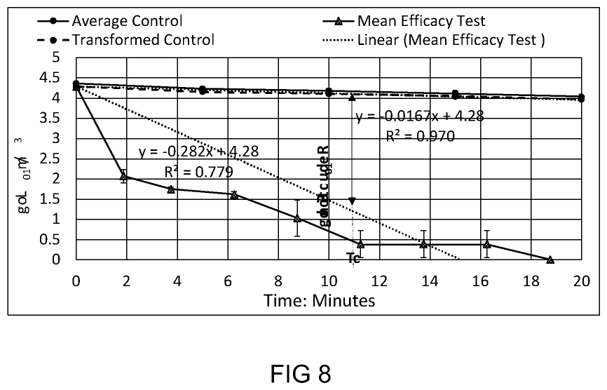

FIG. 8 is a graph of the average log₁₀ concentration of MS2 versus time from Table 3 for the two different lots of Composition N at a 30 second spray time. Each lot tested in triplicate (so the average of 6 test results). As can be seen, the average of the 6 test results for Composition N obtains a 3 log₁₀ reduction in less than approximately 11 minutes.

### Example 3: DPG vs TEG

The four (4) compositions in Table 4 below were tested against S. epidermidis to evaluate the efficacy of DPG versus TEG: TEG Only, DPG Only, TEG-DPG Blend, and TEG-Based. S. epidermidis is a safer, yet equally as relevant as S. aureus, surrogate for a variety of vegetative nosocomial pathogens with potential for airborne spread. Approximately 200 g of product was introduced into the chamber 1 (i.e., the entire can).

TABLE 4

| | TEG Only | DPG Only | TEG-DPG Blend | TEG-Based |
|---|---|---|---|---|
| H2O | 36.8 | 36.8 | 33.8 | 49.03 |
| DPG | 0 | 3 | 3 | 0 |
| TEG | 3 | 0 | 3 | 5.4 |
| CI | 0.2 | 0.2 | 0.2 | 0.1 |
| EtOH | 40 | 40 | 40 | 20.42 |
| Quat | 0 | 0 | 0 | 0.15 |
| Prop | 20 | 20 | 20 | 24.9 |

The DPG Only and TEG-DPG compositions showed a mean ≥3 log₁₀ reduction from a mean baseline titer of 4.56 log₁₀ after a ≤10 minute exposure. The TEG Only composition showed a mean log₁₀ reduction of 1.33 at 10 minutes and 2.35 at 60 minutes from a mean baseline titer of 4.83 log 10. The TEG-Based composition showed a mean log₁₀ reduction of 1.59 at 10 minutes and 2.93 at 60 minutes from a mean baseline titer for 4.36 log₁₀. This data demonstrates that DPG is a more effective microbicide in the air than TEG.

### Example 4: DPG Concentration in Air

The concentration of DPG in the air was determined using Capillary Gas Chromatography (GC) with FID detector for Composition N.

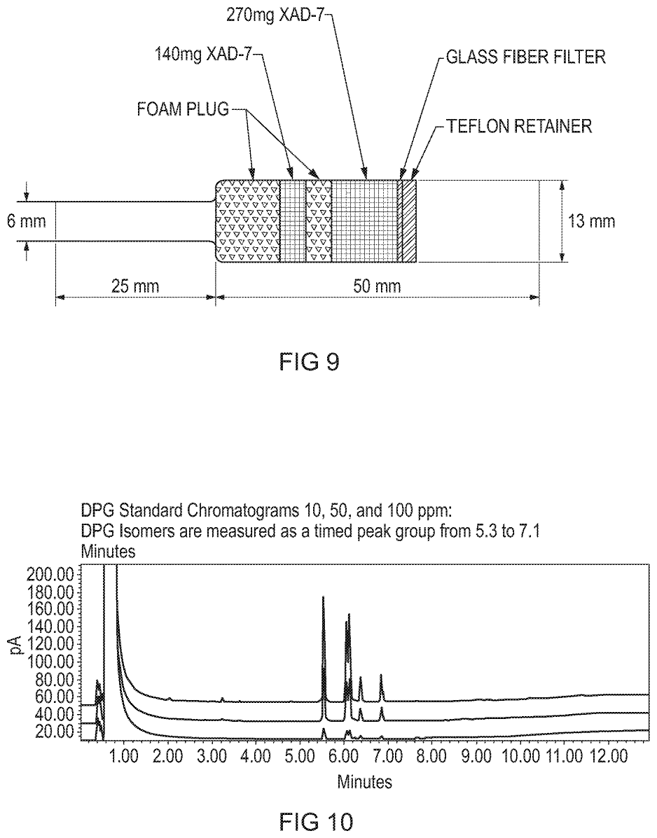

The air samples were collected at the slit-to-agar air sampler 10 of FIG. 2 using a pump, not shown, and custom Occupational Safety and Health Administration (OSHA) Versatile Sampler (OVS) sorbent tubes, particularly configuration 7 (OVS-7), shown in FIG. 9. The pump is calibrated to within +5% of the recommended flow rate.

The custom sorbent tubes had two sections: a 5.0 cm long section with an 11 mm inner diameter and 13 mm outer diameter (o.d.) tapering to a 2.5 cm long section with a 6 mm o.d. A foam plug is inserted into the 13 mm o.d. section until it reaches the junction with the 6 mm o.d. section. 140 mg of hydrophobic organic porous polymer adsorbent, sold under the trade name XAD™-7 by Rohm and Haas, is added to the 13 mm o.d. section to form a layer between the foam plug and a second foam plug is added. An additional 270 mg of polymer adsorbent is added to the 13 mm o.d. section, followed by a glass fiber filler and, finally, a polytetrafluoroethylene (PTFE) retainer. These tubes are currently commercially available as custom products from SKC Inc. (catalog number 226-57), Supelco, and Forest Biomedical.

The 6 mm o.d. end of the custom tube was attached to the pump with flexible tubing. The custom tube was positioned vertically, with the 13 mm o.d. end of the tube pointing obliquely outwards, 76 to 101.6 cm from the floor.

The initial weight of the aerosol can containing composition N was measured and recorded to two decimals as t⁰. Using the gloves 13, the aerosol was sprayed into the testing chamber 1 for 30 seconds with a sweeping motion towards the center of the chamber. The sampling pump was started and samples taken at the intervals specified in the table below. The room temperature, pressure, relative humidity, final aerosol can weight, and sample volume (liters of air) were recorded for each sample and blank comparison test run.

The test, blank comparison, and standard samples were dissolved in 2 mL isopropanol (IPA). The sample tubes were sealed immediately and allowed to extract/desorb for 30 minutes with either manual shaking or use of a sonicator. A portion of the extracted IPA solution was transferred to 4 mL vials suitable for a GC autosampler. At least 2 injections were run for each sample. The GC was calibrated using a three-point external standard curve, with quantitation ranging from 5 to 300 ppm. FIG. 10 is an exemplary calibration chromatogram showing the elution time for the 3 different concentrations of DPG standards, including the peaks for the DPG isomers.

The DPG concentration (in ppm) of the sample was calculated as (Y−C)/M, where Y is the DPG peak area in the GC chromatogram, C is the y intercept from the GC chromatogram, and M is slope of the line from the 3-point primary standard curve. The total PPM of DPG is the sum of the DPG from the absorbent layers in the front and back of the sample tube. The Replicate Analysis Acceptance Criteria (RAAC) is [(Result of Injection 1/Result of Injection 2)−1]*100, and must be within ±10% to be acceptable. The concentration of DPG in the air sample is calculated as:

$$\frac{( Total ppm DPG ) ( DV ) ( 24.46 ) ( 10 \hat{} 6 ) ( g ) ( mg )}{( Liters ) ( DE ) ( MW ) ( 1000 mg ) ( 100 \; 0 μg )} = ppm \times 5.488 = mg / {M}^{3}$$

where.
Total ppm DPG=concentration of DPG in sample
DV=desorption volume=2 mL isopropanol
24.46=Molar volume (liters/mole) at 25 °C and 760 mm HG
Liters=liters of air sample
DE=desorption efficiency=1
MW=molecular weight (g/mole)=134.17
5.488=MwDPG/24.45 (unit conversion factor)

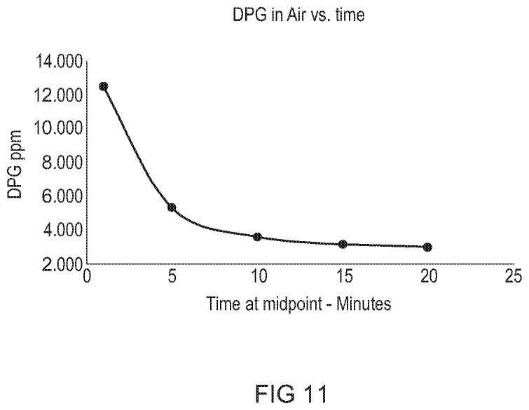

The results are summarized in Table 5 below and FIG. 11.

TABLE 5

| Time Interval | DPG in Sample | | DPG in Air | DPG in Air |
|---|---|---|---|---|
| (in minutes)* | (ppm) | RAAC | (ppm) | (mg/M³) |
| Background | 11.2 | 1.0 | 0.51 | 2.80 |
| 0-2 | 274.1 | −2.4 | 12.49 | 68.56 |
| 4-6 | 116.8 | 2.7 | 5.32 | 29.21 |
| 9-11 | 79.4 | −1.4 | 3.62 | 19.86 |
| 14-16 | 70.1 | 4.2 | 3.20 | 17.53 |
| 19-21 | 66.6 | 8.5 | 3.04 | 16.66 |

*Time is defined as the midpoint of the measurement (e.g., 0-2 minutes = 1, 4-6 minutes = 5, etc)

As can be seen from Table 5 and FIG. 11, the initial concentration of DPG in air measured from 0-2 minutes after spraying the aerosol stopped is approximately 12 ppm. One of ordinary skill in the art will recognize that the concentration of DPG in air will be higher than 12 ppm during the spraying process, particularly towards the end of the spraying process. One of ordinary skill in the art will further recognize the decrease in DPG concentration correlates to the increase in germ kill when FIG. 11 is overlapped with any of FIGS. 5-8. Approximately 10-15 minutes after the spraying is completed, the concentration of DPG in the air achieves an almost steady state of 2-4 ppm, as expected based on the vapor pressure of DPG. This data reinforces that the DPG must be administered in an almost super-saturated vapor state, leading to an instability of the vapor to remain at that concentration. Such scenario leads to rapid partitioning of the vapor onto the external structures of the microbes, helping to create an osmotic shock across the outer bacterial membrane rendering the bacteria non-viable (i.e., killing the bacteria). Lower concentrations of DPG tend to coalesce and settle onto vertical surfaces of treatment areas rather than partitioning onto microbes.

When combined with the data from Table 2, these results confirm Applicant's hypothesis that DPG needs to reach a supersaturated state in order to provide germ kill in the air. These results also confirm that the formulation remains in the air for 2 hours, providing long lasting germ kill and fragrance benefits.

This data also demonstrates that any sheer fragrance aerosolized with the DPG may remain suspended in the air for at least 20 minutes. Additional testing is ongoing to determine the hang time achieved, and specifically how long DPG remain detectible in the air. Applicant expects that DPG will be detectible in the air for at least 60 minutes, preferably up to 2 hours, and more preferably up to 6 hours. If correct, the fragrance intensity of the sheer fragrance is also expected to remain detectible for at least 60 minutes, up to 2 hours, and more preferably up to 6 hours.

### Example 5: Malodour Control

An external agency was used to test the capability of the disclosed compositions to reduce or suppress the perception of cooking malodour in the air based on ASTM Test Method E1697, version 5 (2020), entitled Standard Test Method for Unipolar Magnitude Estimation of Sensory Attributes. The test samples summarized in Table 6 below were sprayed into a large chamber. A panel of 15 fragrance experts smelled the test samples through a hole in the chamber and assigned a malodour score at 0, 10 minutes, 30 minutes, 1 hour, 1.5 hours and 2 hours intervals. The results are also summarized in Table 6 below:

TABLE 6

| Sample | 0 | 10 min | 30 min | 1 hr | 1.5 hr | 2 hr |
|---|---|---|---|---|---|---|
| Malodour Alone | 80 | 93 | 92 | 99 | 90 | 95 |
| Malodour and Water | 95 | 70 | 72 | 68 | 77 | 49 |
| Malodour with an average of 3 different | 83 | 1.2 | 1.7 | 0.7 | 0.8 | 0.7 |
| fragrances in Formulation N* | | | | | | |
| Average of 3 different fragrances in | 0.4 | 0.2 | 0.3 | 0.2 | 0.2 | 0.3 |
| Formulation N without malodour | | | | | | |

*The same 3 fragrances were used in Formulation N with and without malodour.

Water is introduced as a control to demonstrate whether malodour reduction occurs via a mechanical action. As shown in Table 6, water helps reduce the perception of malodour, but not significantly. The averaged results for the disclosed compositions with or without malodour were much closer, demonstrating that the compositions containing 0.07% wt active SME odour neutralizing cationic surfactant and fragrance have reached a threshold of efficacy and maintain efficacy for up to 2 hours. In fact, each of the three fragrances produced greater than 99% reduction in the perception of malodour after 10 minutes and up to 2 hours.

Additional testing was provided comparing compositions containing the same fragrance with both 0.07% wt and 0.21% wt active SME. The fragrance tested was different than the three fragrances tested in Table 6. The malodour score for the 0.07% wt active SME was 6.1 after 10 minutes and 5.7 after 2 hours. The malodour score for the 0.21% wt active SME was 11 after 10 minutes and 12 after 2 hours. Based on these results, it appears that the samples containing 0.21% wt active SME are less successful at masking the malodour than the samples with 0.07% wt active SME. As a result, Applicant believes that the SME may suppress some of the fragrance notes, particularly the top notes, thereby reducing the composition's malodour masking capabilities.

Additional testing of both malodour intensity and fragrance longevity using different SME levels is ongoing to confirm whether the disclosed formulations provide better malodour control with or without SME and, if with, the appropriate concentration to obtain malodour control in air. Applicant believes that additional testing with both 0% wt and higher concentrations of SME and fragrances containing more top notes than bottom notes will provide better results than those obtained from compositions containing fragrances containing more bottom notes than top notes with no SME.

Based on the disclosures herein, below are non-limiting exemplary concepts that may be pursued in non-provisional applications that claim the benefit of this application. These concepts are presented for illustration purposes only and do not in any way limit the scope of the inventive concepts describe herein.
1) A composition comprising
   approximately 7% wt to approximately 43% wt dipropylene glycol;
   approximately 28 wt to approximately 50% wt alcohol;
   approximately 30% wt to approximately 45% wt water; and
   approximately 0.01% wt to approximately 1% wt sheer fragrance, wherein the sheer fragrance comprises approximately 10% wt to approximately 80% wt top notes based on the total weight of the sheer fragrance.
2) The composition of concept 1, wherein the composition is a single phase liquid.
3) The composition of concept 1 or 2, wherein the composition comprises approximately 10% wt to approximately 25% wt dipropyelene glycol.
4) The composition of any one of concepts 1 to 3, wherein the composition comprises approximately 15% wt to approximately 20% wt dipropylene glycol.
5) The composition of any one of concepts 1 to 4, wherein the composition comprises a weight ratio of alcohol:water ranging from approximately 1:1.15 to approximately 1:7.
6) The composition of any one of concepts 1 to 5, wherein the composition is an aerosol composition and comprises a weight ratio of alcohol:water ranging from approximately 1:1.15 to approximately 1:7.
7) The composition of any one of concepts 1 to 6, wherein the composition comprises a weight ratio of alcohol:water ranging from approximately 1.35 to 1.5.
8) The composition of any one of concepts 1 to 7, wherein the composition comprises a weight ratio of dipropylene glycol:water:alcohol ranging from approximately 1:0.8:1.2 to approximately 1:2:2.5. preferably from approximately 1:1.8:2.3 to 1:2:2.5.
9) The composition of any one of concepts 1 to 8, wherein the composition is an aerosol composition and comprises a weight ratio of dipropylene glycol:water:alcohol ranging from approximately 1:0.8:1.2 to approximately 1:2:2.5. preferably from approximately 1:1.8:2.3 to 1:2:2.5.
10) The composition of any one of concept 1 to 9, wherein the composition comprises a weight ratio of dipropylene glycol:water:alcohol of approximately 1:2:2.3.
11) The composition of any one of concept 1 to 10, wherein the composition is an aerosol composition and comprises a weight ratio of dipropylene glycol:water:alcohol of approximately 1:2:2.3.
12) The composition of any one of concept 1 to 9, wherein the composition comprises a weight ratio of dipropylene glycol:water:alcohol of approximately 1:1.8:2.5.
13) The composition of any one of concept 1 to 9 or 12, wherein the composition is an aerosol composition and comprises a weight ratio of dipropylene glycol:water:alcohol of approximately 1:1.8:2.5.
14) The composition of any one of concepts 1 to 13, wherein the composition comprises approximately 0.05% wt to approximately 1.5% wt sheer fragrance.
15) The composition of any one of concepts 1 to 14, wherein the composition comprises approximately 0.1% wt to approximately 1.5% wt sheer fragrance.
16) The composition of any one of concepts 1 to 15, wherein the composition comprises approximately 0.05% wt to approximately 0.75% wt sheer fragrance.
17) The composition of any one of concepts 1 to 16, wherein the composition comprises approximately 0.1% wt to approximately 0.75% wt sheer fragrance.
18) The composition of any one of concepts 1 to 17, wherein the composition comprises approximately 0.1% wt to approximately 0.5% wt sheer fragrance.
19) The composition of any one of concepts 1 to 15, wherein the composition comprises approximately 0.5% wt to approximately 1% wt sheer fragrance.
20) The composition of any one of concepts 1 to 19, wherein the sheer fragrance comprises less than 50% wt volatile solvent based on the total weight of the sheer fragrance.
21) The composition of any one of concepts 1 to 20, wherein the sheer fragrance comprises approximately 15% wt and approximately 65% wt top notes based on the total weight of the sheer fragrance.
22) The composition of any one of concepts 1 to 21, wherein the sheer fragrance comprises approximately 25% wt and approximately 50% wt top notes based on the total weight of the sheer fragrance.
23) The composition of any one of concepts 1 to 22, wherein the sheer fragrance comprises a pro-fragrance.
24) The composition of any one of concepts 1 to 23, wherein the sheer fragrance is substantially free of encapsulated fragrances.
25) The composition of any one of concepts 1 to 24, wherein the composition is substantially free of encapsulated fragrances.
26) The composition of any one of concepts 1 to 25, wherein the composition comprises approximately 0% wt to approximately 5% wt odour neutralizing cationic surfactant.
27) The composition of any one of concepts 1 to 26, wherein the composition comprises approximately 0.1% wt to approximately 5% wt odour neutralizing cationic surfactant.
28) The composition of concept 26 or 27, wherein the composition comprises approximately 1% wt to approximately 2% wt odour neutralizing cationic surfactant.
29) The composition of concept 26 or 27, wherein the composition comprises approximately 0.5% wt to approximately 1% wt odour neutralizing cationic surfactant.
30) The composition of any one of concepts 26 to 29, wherein the odour neutralizing cationic surfactant comprises a quaternary morpholinium alkyl sulfate compound.
31) The composition of concept 30, wherein the quaternary morpholinium alkyl sulfate compound is selected from the group consisting of soyethyl morpholinium ethosulfate, cetethyl morpholinium ethosulfate, N-myristyl-N-methyl morpholinium methyl sulfate, N-oleyl-N-methyl morpholinium methyl sulfate, and combinations thereof.
32) The composition of concept 30 or 31, wherein the quaternary morpholinium alkyl sulfate compound comprises soyethyl morpholinium ethosulfate and/or cetethyl morpholinium ethosulfate.
33) The composition of any one of concepts 30 to 32, wherein the quaternary morpholinium alkyl sulfate compound comprises, consists essentially of, or consists of soyethyl morpholinium ethosulfate.
34) The composition of any one of concepts 30 to 32, wherein the quaternary morpholinium alkyl sulfate compound comprises, consists essentially of, or consists of cetethyl morpholinium ethosulfate.
35) The composition of concept 30 to 31, wherein the quaternary morpholinium alkyl sulfate compound comprises N-myristyl-N-methyl morpholinium methyl sulfate and/or N-oleyl-N-methyl morpholinium methyl sulfate.
36) The composition of any one of concepts 30, 31, or 35, wherein the quaternary morpholinium alkyl sulfate compound comprises, consists essentially of, or consists of N-myristyl-N-methyl morpholinium methyl sulfate.
37) The composition of any one of concepts 30, 31, or 35, wherein the quaternary morpholinium alkyl sulfate compound comprises, consists essentially of, or consists of N-oleyl-N-methyl morpholinium methyl sulfate.
38) The composition of any one of concepts 1 to 37, wherein the composition further comprises a corrosion inhibitor.
39) The composition of any one of concepts 1 to 38, wherein the composition further comprises approximately 0.043% wt to approximately 0.43% wt of the composition of a corrosion inhibitor.
40) The composition of any one of concepts 1 to 39, wherein the composition further comprises approximately 0.043% wt to approximately 0.43% wt of the composition of a pH adjuster.
41) The composition of any one of concepts 1 to 40, wherein the composition further comprises 0.043% wt to 0.43% wt of the composition of a pH adjuster.
42) The composition of concept 40 or 41, wherein the pH adjuster is an inorganic base, an alkanolamine, a buffer, or combinations thereof.
43) The composition of any one of concepts 40 to 42, wherein the pH adjuster is an inorganic base.
44) The composition of any one of concepts 40 to 43, wherein the pH adjuster is an alkali or alkaline hydroxide.
45) The composition of any one of concepts 40 to 44, wherein the pH adjuster is sodium hydroxide, potassium hydroxide, magnesium hydroxide, ammonium hydroxide, or combination thereof.
46) The composition of any one of concepts 40 to 42, wherein the pH adjuster is an alkanolamine.
47) The composition of any one of concepts 40 to 42 or 46, wherein the pH adjuster is monoethanolamine, diethanolamine, triethanolamine, methylethylhydroxypropylhydroxylamine, or combinations thereof.
48) The composition of any one of concepts 40 to 42, 46, or 47, wherein the pH adjuster is monoethanolamine.
49) The composition of any one of concepts 40 to 42, 46, or 47, wherein the pH adjuster is diethanolamine.
50) The composition of any one of concepts 40 to 42, 46, or 47, wherein the pH adjuster is triethanolamine.
51) The composition of any one of concepts 40 to 42, 46, or 47, wherein the pH adjuster is methylethylhydroxypropylhydroxylamine.
52) The composition of any one of concepts 40 to 42, wherein the pH adjuster is a buffer.
53) The composition of any one of concepts 40 to 42 or 52, wherein the pH adjuster is a buffer selected from the group consisting of glycine and sodium hydroxide, ammonia and ammonium chloride, sodium dihydrogen phosphate and sodium hydroxide, or combinations thereof
54) The composition of any one of concepts 1 to 53, wherein a pH of the composition ranges from approximately 9 to approximately 11.
55) The composition of any one of concepts 1 to 54, wherein a pH of the composition ranges from approximately 10 to approximately 10.5.
56) The composition of any one of concepts 1 to 55, wherein the composition comprises 0% wt to approximately 0.28% wt of the composition of a defoaming agent.
57) The composition of any one of concepts 1 to 56, wherein the composition further comprises approximately 0.05% wt to approximately 0.43% wt of the composition of a dye.
58) The composition of any one of concepts 1 to 57, wherein the composition is substantially free of antimicrobials.
59) The composition of any one of concepts 1 to 58, wherein the composition is substantially free of a cationic biocide.
60) The composition of any one of concepts 1 to 59, wherein the composition is substantially free of an halides and halide compounds.
61) The composition of any one of concepts 1 to 60, wherein the composition is substantially free of a chloride and chloride compounds.
62) The composition of any one of concepts 1 to 61, wherein the composition is substantially free of an iodine and iodine compounds.
63) The composition of any one of concepts 1 to 62, wherein the composition is substantially free of phenolic compounds.
64) The composition of any one of concepts 1 to 63, wherein the composition is substantially free of hydrogen peroxide.
65) The composition of any one of concepts 1 to 64, wherein the composition is substantially free of sodium hypochlorite.
66) The composition of any one of concepts 1 to 65, wherein the composition is substantially free of chlorhexidine.
67) The composition of any one of concepts 1 to 66, wherein the composition is substantially free of a quaternary ammonium cationic biocide,
68) The composition of concept 67, wherein the composition is substantially free of benzalkonium chloride.
69) The composition of concept 67, wherein the composition is substantially free of dialkyl dimethyl ammonium chloride.
70) The composition of any one of concepts 1 to 69, wherein the composition is substantially free of silver and silver-containing compounds.
71) The composition of any one of concepts 1 to 70, wherein the composition is substantially free of heavy metals.
72) The composition of any one of concepts 1 to 71, wherein the composition is substantially free of inorganic and organic mercurial.
73) The composition of any one of concepts 1 to 72, wherein the composition is substantially free of an emulsifier.
74) The composition of any one of concepts 1 to 73, wherein the composition is substantially free of a nonionic surfactant.
75) The composition of any one of concepts 1 to 74, wherein the composition is substantially free of an amphoteric surfactant.
76) The composition of any one of concepts 1 to 75, wherein an aroma from the sheer fragrance is detectible in the air for up to approximately 1 hour.
77) The composition of any one of concepts 1 to 76, wherein an aroma from the sheer fragrance is detectible in the air for up to approximately 2 hours.
78) The composition of any one of concepts 1 to 77, wherein an aroma from the sheer fragrance is detectible in the air for up to approximately 6 hours.
79) The composition of any one of concepts 1 to 76, wherein an aroma from the sheer fragrance is detectible in the air to a nose of a subject for up to approximately 1 hour.
80) The composition of any one of concepts 1 to 77, wherein an aroma from the sheer fragrance is detectible in the air to a nose of a subject for up to approximately 2 hours.
81) The composition of any one of concepts 1 to 78, wherein an aroma from the sheer fragrance is detectible in the air to a nose of a subject for up to approximately 6 hours.
82) The composition of any one of concepts 1 to 76, wherein an aroma from the sheer fragrance is detectible in the air to a nose of a human subject for up to approximately 1 hour.
83) The composition of any one of concepts 1 to 77, wherein an aroma from the sheer fragrance is detectible in the air to a nose of a human subject for up to approximately 2 hours.
84) The composition of any one of concepts 1 to 78, wherein an aroma from the sheer fragrance is detectible in the air to a nose of a human subject for up to approximately 6 hours.
85) The composition of any one of concepts 1 to 84, wherein the composition produces a greater than 90% reduction in the perception of malodour after 10 minutes as measured against the malodour alone according to ASTM Test Method E1697.
86) The composition of any one of concepts 1 to 85, wherein the composition produces a greater than 90% reduction in the perception of malodour to a nose of a subject after 10 minutes as measured against the malodour alone according to ASTM Test Method E1697.
87) The composition of any one of concepts 1 to 86, wherein the composition produces a greater than 90% reduction in the perception of malodour to a nose of a person after 10 minutes as measured against the malodour alone according to ASTM Test Method E1697.
88) The composition of any one of concepts 1 to 87, wherein the composition is an air decontamination composition.
89) The composition of any one of concepts 1 to 88, wherein the composition is a malodour control composition.
90) The composition of any one of concepts 1 to 89, wherein the composition is air decontamination and malodour control composition.
91) An aerosol product comprising an aerosol can comprising approximately 70% wt to approximately 80% wt of the composition of any one of concepts 1 to 90, and approximately 20% wt to approximately 30% wt of a propellant capable of producing at least 60 psig [414 kPa] pressure, both weight percentages based upon the combined weight of the composition and the propellant.
92) The aerosol product of concept 91, wherein aerosol product produces a spray having an average particle size ranging from approximately Dv[50]=45 microns to approximately Dv[50]=90 microns as measured using a laser diffractometer, preferably from approximately Dv[50]=50 microns to approximately Dv[50]=60 microns.
93) The aerosol product of concept 91 or 92, wherein the aerosol product further comprises a valve assembly attached to the can, the valve assembly comprising a 2-piece mechanical breakup (MB) nozzle with swirl chamber or break up bar, the nozzle comprising two stem orifices, each having a diameter ranging from approximately 0.91 mm to approximately 1.12 mm.
94) The aerosol product of any one of concepts 91 to 93, wherein the aerosol product produces a greater than 90% reduction in the perception of malodour after 10 minutes as measured against the malodour alone according to ASTM Test Method E1697.
95) The aerosol product of any one of concepts 91 to 94, wherein an aroma from the sheer fragrance is detectable in the air for up to approximately 1 hour.
96) The aerosol product of any one of concepts 91 to 95, wherein an aroma from the sheer fragrance is detectable in the air for preferably up to approximately 2 hours.
97) The aerosol product of any one of concepts 91 to 96, wherein an aroma from the sheer fragrance is detectable in the air for up to approximately 6 hours.
98) The aerosol product of any one of concepts 91 to 95, wherein an aroma from the sheer fragrance is detectible in the air to a nose of a subject for up to approximately 1 hour.
99) The aerosol product of any one of concepts 91 to 96, wherein an aroma from the sheer fragrance is detectible in the air to a nose of a subject for up to approximately 2 hours.
100) The aerosol product of any one of concepts 91 to 97, wherein an aroma from the sheer fragrance is detectible in the air to a nose of a subject for up to approximately 6 hours.
101) The aerosol product of any one of concepts 91 to 95, wherein an aroma from the sheer fragrance is detectible in the air to a nose of a human subject for up to approximately 1 hour.
102) The aerosol product of any one of concepts 91 to 96, wherein an aroma from the sheer fragrance is detectible in the air to a nose of a human subject for up to approximately 2 hours.
103) The aerosol product of any one of concepts 91 to 97, wherein an aroma from the sheer fragrance is detectible in the air to a nose of a human subject for up to approximately 6 hours.
104) The aerosol product of any one of concepts 91 to 103, wherein the aerosol can is connected to the valve assembly by a mounting cup.
105) The aerosol product of any one of concepts 91 to 104, wherein the valve assembly comprises an actuator comprising a 2-piece mechanical break-up (MB) nozzle with swirl chamber.
106) The aerosol product of any one of concepts 91 to 105, wherein the MB nozzle comprises two stem orifices having a diameter ranging from approximately 0.036 inches [0.91 mm] to approximately 0.044 inches [1.12 mm].
107) The aerosol product of any one of concepts 91 to 106, wherein the MB nozzle comprises two stem orifices having a diameter ranging from approximately 0.038 inches [0.97 mm] to approximately 0.042 inches [1.07 mm].
108) The aerosol product of any one of concepts 91 to 107, wherein the propellant is a blend of propane and isobutane.
109) The aerosol product of any one of concepts 91 to 108, wherein the aerosol product is substantially free of dimethyl ether propellant.
110) The aerosol product of any one of concepts 91 to 109, wherein the MB nozzle has an actuator orifice with a diameter ranging from approximately 0.016 inches [0.41 mm] to approximately 0.022 inches [0.56 mm].
111) The aerosol product of any one of concepts 91 to 110, wherein the MB nozzle has an actuator orifice with a diameter ranging from approximately 0.017 inches [0.43 mm] to approximately 0.021 inches [0.53 mm].
112) The aerosol product of any one of concepts 91 to 111, wherein the MB nozzle has an actuator orifice with a diameter ranging from approximately 0.018 inches [0.46 mm] to approximately 0.020 inches [0.51 mm].
113) The aerosol product of any one of concepts 91 to 112, wherein the MB nozzle has a stem vapor tap with a diameter ranging from approximately 0.016 inches [0.41 mm] to approximately 0.025 inches [0.64 mm].
114) The aerosol product of any one of concepts 91 to 113, wherein the MB nozzle has a stem vapor tap with a diameter ranging from approximately 0.017 inches [0.43 mm] to approximately 0.024 inches [0.61 mm].
115) The aerosol product of any one of concepts 91 to 114, wherein the MB nozzle has a stem vapor tap with a diameter ranging from approximately 0.018 inches [0.46 mm] to approximately 0.023 inches [0.58 mm].
116) The aerosol product of any one of concepts 91 to 115, wherein the MB nozzle has a stem vapor tap with a diameter ranging from approximately 0.019 inches [0.48 mm] to approximately 0.022 inches [0.56 mm].
117) The aerosol product of any one of concepts 91 to 116, wherein the 2-piece MB nozzle produces a spray having a particle size ranging from approximately Dv[50]=45 microns to approximately Dv[50]=90 microns on average as measured using a laser diffractometer.
118) The aerosol product of any one of concepts 91 to 117, wherein the 2-piece MB nozzle produces a spray having a particle size ranging from approximately Dv[50]=50 microns to approximately Dv[50]=60 microns on average as measured using a laser diffractometer.
119) The aerosol product of any one of concepts 91 to 118, wherein the 2-piece MB nozzle produces a spray having a particle size ranging from approximately Dv[50]=45 microns to approximately Dv[50]=55 microns on average as measured using a laser diffractometer.
120) The aerosol product of any one of concepts 91 to 119, wherein the 2-piece MB nozzle produces a spray having a volume moment mean ranging from Dv[4,3]=55 microns to Dv[4,3]=75 microns on average as measured using a laser diffractometer.
121) The aerosol product of any one of concepts 91 to 120, wherein the 2-piece MB nozzle produces a spray having a volume moment mean ranging from Dv[4,3]=60 microns to Dv[4,3]=70 microns on average as measured using a laser diffractometer.
122) The aerosol product of any one of concepts 91 to 121, wherein a ratio of the vapor tap to exit orifice ranges from approximately 2:1 to 1:2, preferably from approximately 1:0.9 to 1:1.1, and more preferably approximately 1:1.
123) A method of reducing or suppressing the perception of malodour in the air, the method comprising generating an aerosolized spray from the aerosol product of any one of concepts 91 to 122.
124) A method of reducing transmission of germs from the air to surfaces, the method comprising generating an aerosolized spray from the aerosol product of any one of concepts 91 to 122.
125) A method of prolonging the perception of a sheer fragrance in the air, the method comprising generating an aerosolized spray from the aerosol product of any one of concepts 91 to 122.
126) The method of any one of concepts 123 to 125, wherein generating the aerosolized spray introduces into air between approximately 1.1 g/second to approximately 3 g/second of the composition of any one of concepts 1 to 90.
127) The method of any one of concepts 123 to 126, wherein generating the aerosolized spray introduces into air between approximately 1.5 g/second to approximately 2.5 g/second of the composition of any one of concepts 1 to 90.
128) The method of any one of concepts 123 to 127, wherein the method further disinfects the air in approximately 0.1 minutes to approximately 5 minutes.
129) The method of any one of concepts 123 to 129, wherein the method provides a 3 log₁₀ reduction of aerosolized non-enveloped viruses, like MS2, in 10-15 minutes.
130) The method of any one of concepts 123 to 129, wherein the aerosolized spray comprises between approximately 5 ppm to approximately 30 ppm dipropylene glycol in vapor form.
131) The method of any one of concepts 123 to 130, wherein the method supersaturates the air with a DPG vapor.
132) The method of any one of concepts 123 to 131, wherein the air occupies less than or equal to 25 m³.
133) The method of any one of concepts 123 to 132, wherein the aerosolized spray introduces approximately 5 ppm to approximately 30 ppm of dipropylene glycol vapor into the air for approximately 10 to approximately 30 seconds, the air occupying a volume less than or equal to 25 m³.
134) The method of any one of concepts 123 to 133, wherein the aerosolized spray introduces approximately 9 ppm to approximately 12 ppm of dipropylene glycol vapor into the air for approximately 10 to approximately 30 seconds, the air occupying a volume less than or equal to 25 m³.
135) The method of any one of concepts 123 to 134, wherein the aerosolized spray introduces approximately 1.1 g/second to approximately 3.0 g/second of the composition of any one of concepts 1 to 90 into the air for approximately 10 to approximately 60 seconds, the air occupying a volume less than or equal to 25 m³.
136) The method of any one of concepts 123 to 135, wherein the aerosolized spray introduces approximately 1.5 g/second to approximately 2.5 g/second of the composition of any one of concepts 1 to 90 into the air for approximately 10 to approximately 60 seconds, the air occupying a volume less than or equal to 25 m³.
137) The method of any one of concepts 123 to 136, wherein the aerosolized spray is generated in one continuous 10 to 60 second spray.
138) The method of any one of concepts 123 to 136, wherein the aerosolized spray is generated in multiple consecutive sprays that combine to total 10 to 60 seconds of spray in a total time of less than 15 to 40 seconds.
139) The method of any one of concepts 123 to 136, wherein the aerosolized spray is generated for approximately 10 to approximately 45 seconds.
140) The method of any one of concepts 123 to 136, wherein the aerosolized spray is generated for approximately 10 to approximately 30 seconds.
141) The method of any one of concepts 123 to 140, wherein the aerosolized spray is generated using the valve assembly of concept 93.
142) The method of concept 141, wherein an actuator on the valve assembly is pressed for approximately 10 to approximately 60 seconds.
143) The method of concept 141 or 142, wherein an actuator on the valve assembly is pressed for approximately 15 to approximately 40 seconds.
144) The method of any one of concepts 123 to 143, wherein the aerosolized spray has a particle size ranging from approximately Dv[50]=45 microns to approximately Dv[50]=90 microns, as measured using laser diffraction.
145) The method of any one of concepts 123 to 144, wherein the aerosolized spray has a particle size ranging from approximately Dv[50]=50 microns to approximately Dv[50]=60 microns, as measured using laser diffraction.
146) The method of any one of concepts 123 to 144, wherein the aerosolized spray has a volume moment mean ranging from Dv[4,3]=55 microns to Dv[4,3]=75 microns on average as measured using a laser diffractometer.
147) The method of any one of concepts 123 to 146, wherein the aerosolized spray has a volume moment mean ranging from Dv[4,3]=60 microns to Dv[4,3]=70 microns on average as measured using a laser diffractometer.
148) The method of any one of concepts 123 to 147, wherein the method produces a greater than 90% reduction in the perception of malodour after 10 minutes as measured against the malodour alone according to ASTM Test Method E1697.
149) The method of any one of concepts 123 to 148, wherein the method produces a greater than 90% reduction in the perception of kitchen malodour after 10 minutes as measured against the malodour alone according to ASTM Test Method E1697.
150) The method of any one of concepts 123 to 149, wherein the method produces a greater than 90% reduction in the perception of malodour after 2 hours as measured against the malodour alone according to ASTM Test Method E1697.
151) The method of any one of concepts 123 to 150, wherein the method produces a greater than 90% reduction in the perception of kitchen malodour after 2 hours as measured against the malodour alone according to ASTM Test Method E1697.
152) The method of any one of concepts 123 to 151, wherein an aroma from the sheer fragrance is detectible in the air for up to approximately 1 hour.
153) The method of any one of concepts 123 to 152, wherein an aroma from the sheer fragrance is detectible in the air for up to approximately 2 hours.
154) The method of any one of concepts 123 to 153, wherein an aroma from the sheer fragrance is detectible in the air for up to approximately 6 hours.
155) The method of any one of concepts 123 to 152, wherein an aroma from the sheer fragrance is detectible in the air to a nose of a subject for up to approximately 1 hour.
156) The method of any one of concepts 123 to 153, wherein an aroma from the sheer fragrance is detectible in the air to a nose of a subject for up to approximately 2 hours.
157) The method of any one of concepts 123 to 154, wherein an aroma from the sheer fragrance is detectible in the air to a nose of a subject for up to approximately 6 hours.
158) The method of any one of concepts 123 to 152, wherein an aroma from the sheer fragrance is detectible in the air to a nose of a human subject for up to approximately 1 hour.
159) The method of any one of concepts 123 to 153, wherein an aroma from the sheer fragrance is detectible in the air to a nose of a human subject for up to approximately 2 hours.
160) The method of any one of concepts 123 to 154, wherein an aroma from the sheer fragrance is detectible in the air to a nose of a human subject for up to approximately 6 hours.
161) The method of any one of concepts 123 to 160, wherein the method provides a greater than 90% reduction in the perception of malodour after 10 minutes as measured against the malodour alone according to ASTM Test Method E1697.
162) The method of any one of concepts 123 to 161, wherein the method provides a greater than 90% reduction in the perception of malodour to a nose of a subject after 10 minutes as measured against the malodour alone according to ASTM Test Method E1697.
163) The method of any one of concepts 123 to 162, wherein the method provides a greater than 90% reduction in the perception of malodour to a nose of a person after 10 minutes as measured against the malodour alone according to ASTM Test Method E1697.
164) The method of any one of concepts 123 to 163, wherein the DPG vapor remains detectible in the air for 20 minutes, preferably for 1 hour, more preferably for 2 hours, and most preferably for 6 hours.
165) The method of concept 164, wherein the DPG vapor is detected using Capillary Gas Chromatography (GC) with FID detector.

The foregoing description of various aspects of the invention has been presented for purposes of illustration and description. Embodiments and/or features therein may be freely combined with one another. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

What is claimed is:

1. An air treatment method, the method comprising (a) sanitizing or disinfecting air and (b) suspending a sheer fragrance in air for at least 20 minutes by generating an aerosolized spray from an aerosol product to produce between approximately 5 ppm and approximately 30 ppm dipropylene glycol in vapor form,
wherein the aerosol product comprises an aerosol can comprising approximately 70% wt to approximately 80% wt of a composition and approximately 20% wt to approximately 30% wt of a propellant capable of producing at least 60 psig [414 kPa] pressure, both weight percentages based upon the combined weight of the composition and the propellant,
wherein the composition comprises, based on the weight of the composition,
approximately 7% wt to approximately 43% wt dipropylene glycol;
approximately 28 wt to approximately 50% wt alcohol;
approximately 30% wt to approximately 45% wt water; and
approximately 0.2% wt to approximately 2% wt sheer fragrance,
wherein the sheer fragrance comprises approximately 10% wt to approximately 80% wt top notes based on the total weight of the sheer fragrance.

2. The air treatment method of claim 1, wherein the composition comprises approximately 0.5% wt to approximately 1% wt sheer fragrance.

3. The air treatment method of claim 1, wherein the method further reduces or suppresses the perception of malodour in the air.

4. The air treatment method of claim 1, wherein the composition further comprises approximately 0.1% wt to approximately 5% wt odour neutralizing cationic surfactant.

5. The air treatment method of claim 1, wherein the composition further comprises approximately 0.1% wt to approximately 5% wt of an odour trapping oligosaccharide selected from the group consisting of alpha-cyclodextrin, beta-cyclodextrin, gamma-cyclodextrin, methyl esters of beta-cyclodextrin, hydroxypropyl beta-cyclodextrin, methylated beta-cyclodextrin, hydroxethyl alpha-cyclodextrin, hydroxyethyl beta-cyclodextrin, or any combination thereof.

6. The air treatment method of claim 1, wherein the composition further comprises a pH adjuster to adjust the composition to a pH ranging from approximately 9 to approximately 11.

7. The air treatment method of claim 6, wherein the pH adjuster is monoethanolamine.

8. The air treatment method of claim 6, wherein the pH ranges from approximately 10 to approximately 10.5.

9. The air treatment method of claim 1, wherein the sheer fragrance comprises less than approximately 50% wt volatile solvent based on the total weight of the fragrance.

10. The air treatment method of claim 1, wherein the sheer fragrance further comprises a profragrance.

11. The air treatment method of claim 1, wherein the composition is substantially free of antimicrobial actives.

12. The air treatment method of claim 11, wherein the antimicrobial actives comprise cationic biocides, halides, halide-containing compounds, silver, silver-containing compounds, phenols, hydrogen peroxide, sodium hypochlorite, chlorhexidine, quaternary ammonium compounds, or any combinations thereof.

13. The air treatment method of claim 1, wherein, other than the odour neutralizing cationic surfactant, the composition is substantially free of surfactants.

14. The air treatment method of claim 1, wherein the aerosol product further comprises a valve assembly attached to the can, the valve assembly comprising a 2-piece mechanical breakup (MB) nozzle with swirl chamber or break up bar, the nozzle comprising two stem orifices, each having a diameter ranging from approximately 0.91 mm to approximately 1.12 mm.

15. The air treatment method of claim 1, wherein an aroma from the sheer fragrance is detectible in the air for up to approximately 1 hour.

16. The air treatment method of claim 1, wherein a hang time of the composition as determined by the DPG concentration in the air is at least 20 minutes.

17. The air treatment method of claim 1, wherein the method provides the greater than or equal to 3 log₁₀ reduction of aerosolized bacteria in the air in approximately 0.1 minutes to approximately 5 minutes.

18. The air treatment method of claim 1, wherein the method produces a greater than 90% reduction in the perception of malodour after 10 minutes as measured against the malodour alone according to ASTM Test Method E1697.

19. The air treatment method of claim 1, wherein the sheer fragrance is detectible in the air for up to approximately 2 hours.

20. An air treatment method, the method comprising:
(a) reducing a concentration of aerosolized bacteria in air by greater than or equal to 3 log₁₀ in approximately 0.1 minutes to approximately 5 minutes,
(b) suspending a sheer fragrance in air for at least 20 minutes, and
(c) reducing or suppressing the perception of a malodour in the air by greater than 90% after 10 minutes as measured against the malodour alone according to ASTM Test Method E1697, by supersaturating air with dipropylene glycol vapor from an aerosol product.
